(19) United States
(12) Patent Application Publication
(10) Pub. No.: US 2022/0354786 A1
(43) Pub. Date: Nov. 10, 2022

(54) SUSPENSION COMPRISING A PROTEIN PARTICLE SUSPENDED IN A NON-AQUEOUS VEHICLE

(71) Applicant: NOVALIQ GMBH, Heidelberg (DE)

(72) Inventors: Wolfgang FRIESS, Seeshaupt (DE); Madlen WITT, Mannheim (DE); Christoph MARSCHALL, Stuttgart (DE)

(21) Appl. No.: 17/751,512

(22) Filed: May 23, 2022

Related U.S. Application Data
(63) Continuation of application No. PCT/EP2020/083683, filed on Nov. 27, 2020.

(30) Foreign Application Priority Data
Nov. 27, 2019 (EP) 19211925.3

(51) Int. Cl. A61K 9/10; C07K 16/22; A61K 47/26; A61K 47/06

(52) U.S. Cl. CPC A61K 9/10; C07K 16/22; A61K 47/26; A61K 47/06; C07K 2317/24

(57) ABSTRACT

The present invention provides for a suspension formulation comprising a protein particle suspended in a non-aqueous vehicle, wherein the particle comprises a protein and a stabilizing agent, and wherein the residual water content of the suspended protein particle is less than 1.0 wt % based on total weight of the particle.

### CROSS-REFERENCE TO RELATED APPLICATIONS

This application is a continuation of International Application No. PCT/EP2020/083683, filed on Nov. 27, 2020, which claims priority to and the benefit of European Application No. 19211925.3, filed on Nov. 27, 2019, the contents of each of which are hereby incorporated by reference in their entireties.

### DESCRIPTION

### Background of the Invention

Drug products based on proteins and antibodies as active ingredients are typically formulated as aqueous solutions or lyophilizates, which require reconstitution prior to use and administration. Protein instability is however prevalent in aqueous solutions, and as a result such products have limited shelf-life and/or requiring the development of complex cold-chain solutions. The alternative approach is the provision of the protein drugs as a lyophilized (i.e. freeze-dried) solid power form, but lyophilizates require careful and accurate reconstitution under sterile conditions in an aqueous media before use and thus are generally less convenient for patient and health-care provider use. The reconstitution step itself may trigger aggregation if the pH or temperature of the aqueous medium is suboptimal, the time allowed for rehydration is too short or the vial is too aggressively shaken during the dissolving step. The propensity for waste is also higher, as failure to properly dissolve the lyophilized antibody product within the recommended time period usually requires for the sample to be discarded.

Ready-to-use liquid formulations would generally be preferred by the users, due to the ease of preparation for administration. As an alternative to aqueous formulations, protein suspensions in non-aqueous carriers and vehicles have been described.

For example, WO2013/110621 describes the formulation of proteins and polypeptides in semifluorinated alkanes. WO2015/011199 describes antibodies suspended in semifluorinated alkanes, also as a means for formulating these types of compounds. It is described that formulating proteins, polypeptides and antibodies in semifluorinated alkanes provides against the degradation or aggregation of such molecules.

There is still a need, however for the provision of non-aqueous suspension formulations of protein particles, i.e. particles comprising a protein and one or more excipient(s), such as stabilizing agent, which are suitable for storage and resistant to changes in storage conditions, such as fluctuations in elevation in storage temperatures. There is furthermore a need for the provision of suspensions of protein particles comprising a protein/polypeptide and one or more excipient(s) having stable suspension characteristics, such as particle size, and particle size stability which allows for the injection of the suspension, also subsequently to storage.

It is therefore an object of the present disclosure to provide for non-aqueous protein particle suspension formulations which may be are stable for storage, and for injection. Another object is to provide for a process or method for the preparation of said non-aqueous protein particle suspension formulations. Further objects of the invention will be clear on the basis of the following description of the invention, examples and claims.

### SUMMARY OF THE INVENTION

In a first aspect, the present invention relates a method of preparing a suspension formulation comprising a protein particle and a non-aqueous vehicle, said method comprising the steps of: a) providing an aqueous solution comprising a protein and a stabilizing agent, b) removing the water from said aqueous solution comprising the protein and the stabilizing agent to obtain solid protein particles, c) further drying the protein particles obtained in step b) so as to obtain protein particles with a residual water content of less than 0.5 wt % based on the total weight of the particle, and d) suspending the protein particles of step c) in the non-aqueous vehicle, and optionally; e) homogenizing the suspension formulation, preferably by high-shear homogenization, milling, or ultrasonication.

The present invention also relates to compositions comprising a protein particle obtainable by the method of the invention.

In a further aspect, the invention relates to a suspension formulation comprising a protein particle suspended in a non-aqueous vehicle, wherein the particle comprises a protein and a stabilizing agent, and wherein the residual water content of the suspended protein particle is less than 0.5 wt % based on total weight of the particle.

In yet a further aspect, the invention provides for the use of the suspension formulation as described for therapeutic and/or diagnostic applications.

### BRIEF DESCRIPTION OF THE DRAWINGS

FIG. 1 depicts from left to right the particle size distributions of suspension formulations of protein particles comprising lysozyme and sucrose (relative ratio 50:50) in non-aqueous vehicle as follows: 5a (F6H8; particles non-dried), 5b (F6H8; particles vacuum dried), 6a (EO; particles non-dried), 6b (EO; particles vacuum dried) as described in Table 1. These suspension formulations were prepared by homogenization in an ice-cooled ultrasound bath.

FIG. 2 depicts from left to right the particle size distributions of suspension formulations of protein particles comprising model monoclonal antibody mAb and sucrose (relative ratio 50:50; total solid content (TSC) 100 mg/mL) in non-aqueous vehicles as follows: 7a (F4H5; particles non-dried)), 7b (F4H5, vacuum dried particles), 8a (F6H8, particles non-dried), 8b (F6H8, vacuum dried particles), 9a (ethyl oleate, particles non-dried), 9b (ethyl oleate, vacuum dried particles), 10a (medium chain triglyceride, particles non-dried), 10b (medium chain triglyceride, vacuum dried particles). These suspension formulations were prepared in an ice-cooled ultrasound bath.

FIG. 3 depicts from left to right, the particle size distribution of suspension formulations of protein particles comprising model monoclonal antibody mAb and stabilizing agent sucrose (mAb:Suc 50:50; TSC=100 mg/ml): 11a (F6H8, particles non-dried), 11b (F6H8, vacuum dried particles), 12a (MCT, particles non-dried), 12b (MCT, vacuum drying). Suspension formulations were homogenized utilizing a high-shear homogenizer.

FIG. 4 depicts from left to right, the particle size distribution of suspension formulations of protein particles comprising bevacizumab (beva) and stabilizing agent sucrose (beva:Suc 50:50; TSC=100 mg/ml): 14a (F6H8, particles non-dried), 14b (F6H8, vacuum dried particles), 15a (EO, particles non-dried), 15b (EO, vacuum drying). These suspension formulations were prepared in an ultrasound bath for homogenization.

FIG. 5 depicts the particle size distribution of suspension formulations, from left to right: suspension formulation 8a prepared from protein particles which were not subjected to vacuum drying (F6H8, mAb:Suc 50:50; TSC=100 mg/ml), after storage for 6 months at 5° C., suspension formulation 8b (F6H8, mAb:Suc 50:50; TSC=100 mg/ml) prepared from vacuum-dried protein particles after 6 months storage at 5° C., suspension formulation 8a after 6 months storage at 25° C., and suspension formulation 8b after 6 months storage at 25° C.

FIGS. 6A and 6B depict particle size distributions of suspension formulations of protein particles comprising a model mAb and sucrose in F4H5 as the liquid vehicle after storage at 0, 1, 3 and 6 months storage at 40° C. FIG. 6A depicts the particle size distributions of suspension formulation 7a (mAb:Suc 50:50; TSC=100 mg/ml) which are prepared from particles which have not been subjected to vacuum drying, and which contained about 4.2 wt % residual water content. FIG. 6B depicts the particle size distributions of suspension formulation 7b (mAb:Suc 50:50; TSC=100 mg/ml) which comprise about 0.1 wt % residual water content. Particle size distributions are represented in these figures as D5 (● solid circle), D10 (0 hollow circle), D50 (♦ solid triangle), D90 (Δ hollow triangle) and D95 (▪ solid square) mean diameter particle size values which were determined using laser diffraction.

FIGS. 7A and 7B depict particle size distributions of suspension formulations of suspension formulations of protein particles comprising a model mAb and sucrose in F6H8 as the liquid vehicle after 0, 1, 3 and 6 months storage at 40° C. FIG. 7A depicts the particle size distributions of suspension formulation 8a (mAb:Suc 50:50; TSC=100 mg/ml), which was prepared from particles which were not subjected to vacuum drying, and which contained about 4.2 wt % residual water content. FIG. 7B depicts the particle size distribution of suspension formulation 8b (mAb:Suc 50:50; TSC=100 mg/mL) which comprises only about 0.1 wt % residual water content. Particle size distributions are represented in these figures as D5 (● solid circle), D10 (◯ hollow circle), D50 (♦ solid triangle), D90 (Δ hollow triangle) and D95 (▪ solid square) mean diameter particle size values which were determined using laser diffraction.

FIG. 8 depicts XRD spectra of the particles of formulation 8a (F6H8, mAb:Suc 50:50; TSC=100 mg/ml, prepared without vacuum-drying) after storage at 5° C., 25° C., and 40° C. for 6 months (spectra A), and XRD spectra of the protein particles of formulation 8b (mAb:Suc 50:50; TSC=100 mg/mL, particles prepared with vacuum drying) after storage at 5° C., 25° C., and 40° C. for 6 months (spectra B).

FIGS. 9A, 9B, 9C depict the particle size stability of formulations prepared with spray-dried and vacuum dried particles comprising lysozyme and trehalose and F6H8 as the liquid vehicle, after 0, 1, 3, 6 and 12 months storage at 40° C. FIG. 9A depicts particle size distributions of suspension formulation 2 (Lys:Tre 70:30; TSC=300 mg/ml). FIG. 9B depicts particle size distributions of suspension formulation 3 (PS20 containing Lys:Tre 70:30 formulation; TSC=100 mg/ml). FIG. 9C depicts particle size distribution of suspension formulation 4 (Lys:Tre 50:50; TSC=100 mg/ml). Particle size distributions are represented in these figures as D5 (● solid circle), D10 (◯ hollow circle), D50 (♦ solid triangle), D90 (Δ hollow triangle) and D95 (▪ solid square) particle size values (mean diameter) which were determined using laser diffraction.

FIG. 10 depicts the resuspendability of suspension formulations, from left to right of each group, formulations 1a, 1b, 1c, 1d (Lys:Tre 70:30; TSC=100 mg/ml, liquid vehicle F4H5, F6H8, EO, MCT respectively) stored over a 12-month period at 5° C., 25° C., and 40° C. Resuspendability was determined using a vertical shaker (vertical rotation at 25 rpm) and is based on time (s) needed for resuspension as determined by visual inspection.

FIG. 11 depicts the resuspendability of suspension formulations, from left to right for each group, the formulations 5a, 5b, 6a, 6b (Lys:Suc 50:50; TSC=100 mg/ml; 5a (F6H8; particles non-dried), 5b (F6H8; vacuum dried), 6a (EO; particles non-dried), 6b (EO; vacuum dried) as described in Table 1)). FIG. 11A depicts resuspendability based on time required for resuspension based on vertical rotation as determined by visual inspection.; FIG. 11B depicts resuspendability based on shaking method, i.e. the frequency of shaking needed for resuspension. The horizontal line depicted at 5 Hz in FIG. 11 B describes the frequency an average person uses for this operation.

FIG. 12 depicts the resuspendability, from each group from left to right of suspension formulations 7a, 7b, 8a, 8b, 9b, 10b (mAb:Suc 50:50; TSC=100 mg/ml) as described in Table 1, after storage for 1 month at 40° C., for 3 months at 40° C. or for 6 months at 5° C., 25° C. and 40° C. Resuspendability is based on frequency of shaking needed for resuspension. The horizontal line depicted at 5 Hz in B describes the frequency an average person uses for this operation.

FIGS. 13A and 13 B depict the maximum injection force (or gliding force) required for injection to obtain a volume flow of 0.1 ml/s using a 27G needle and 1 mL syringe, of suspension formulations comprising lysozyme-trehalose containing particles (lysozyme trehalose 70:30; TSC=100 mg/ml), formulations 1a, 1b, 1c, and 1d, as described in Table 1, over a period of 12 months storage at 40° C. FIG. 13A depicts the injectability test results for formulation 1a (vehicle F4H5), 1b (vehicle F6H8), and FIG. 13B depicts injectability test results for formulation 1c (vehicle EO); 1d (vehicle MCT).

FIG. 14 depicts the glide force profile of formulations 2 (upper curve) and 3 (lower curve) after 12 months of storage at 40° C., as based on force required for injection at a volume flow of 0.1 ml/s using a 27G needle and 1 mL syringe.

FIGS. 15A and 15B depict the maximum injection force (or gliding force) required for injection to obtain a volume flow of 0.1 ml/s using a 27G needle and 1 mL syringe, of suspension formulations with protein particles comprising model mAb-sucrose, 7a, 7b, (F4H5, mAb:Suc 50:50; TSC=100 mg/ml), and 8a, 8b (F6H8, mAb:Suc 50:50; TSC=100 mg/ml), as described in Table 1 stored over a period of 6 months at 40° C. FIG. 15A depicts the injectability test results for the formulations prepared with protein particles which were not subjected to the additional vacuum drying step, 7a, and 8a, the particles containing about 4.2 wt % of residual water content. FIG. 15B, depicts the injectability results for the suspension formulations 7b and 8b prepared with protein particles which were vacuum dried after spray drying, and having a residual water content of about 0.1 wt % relative to weight of the particle.

FIG. 16 depicts the glide force profile of suspension formulations 10b (upper curve) and 9b (lower curve) as described in Table 1, after 6 months of storage at 40° C., as based on force required for injection at a volume flow of 0.1 ml/s using a 27G needle and 1 mL syringe.

FIG. 17 depicts the maximum injection force (or gliding force) required for injection to obtain a volume flow of 0.1 ml/s using a 27G needle and 1 mL syringe, of suspension formulations with protein particles comprising bevacizumab-sucrose, 14a, 14b, (F6H8, beva:suc 50:50, TSC—100 mg/mL), and 15a, 15b (EO, beva:suc 50:50, TSC—100 mg/mL) after storage at 3 months (14a, 14b) and after 6 months period of 6 months at 40° C. (14a, 14b, 15a, 15b). Depicted, from left to right of the graph are the measurements for: 14a, 14b//14a, 14b, 15a, 15b.

### DETAILED DESCRIPTION OF THE INVENTION

The inventors surprisingly found that a composition comprising protein particles, comprising a protein and a stabilizer, wherein the protein particles are suspended in a non-aqueous vehicle, shows highly advantageous properties, if the protein particles are prepared in accordance with the method of the invention.

In particular, compositions comprising protein particles that were obtained by consecutive two different drying steps resulted in improved chemical and physical stability, which in combination with an improved size distribution and easy redispersibility allows for an improved injection application via syringe.

Accordingly, in a first aspect, the present invention relates to a method of preparing a suspension formulation comprising a protein particle and a non-aqueous vehicle, said method comprising the steps of:
a) providing an aqueous solution comprising a protein and a stabilizing agent,
b) removing the water from said aqueous solution comprising the protein and the stabilizing agent to obtain solid protein particles,
c) further drying the protein particles obtained in step b) so as to obtain protein particles with a residual water content of less than 0.5 wt % based on the total weight of the particle, and
d) suspending the protein particles of step b) in the non-aqueous vehicle, and optionally;
e) homogenizing the suspension formulation, preferably by high-shear homogenization, milling, or ultrasonication;
wherein the protein particle comprises a protein and a stabilizing agent, and wherein the non-aqueous vehicle comprises a semifluorinated alkane, a medium chain triglyceride (MCT), ethyl lactate, ethyl oleate or mixtures thereof.

The method of the invention is suitable to produce protein particle suspensions, with improved surprising properties. The method essentially includes two drying steps. A first step b) removes the water from an aqueous solution comprising the protein and a stabilizing agent to obtain protein particles, to result in a residual water content of less than 5 wt % or less than 3 wt %, or to result in a residual water content in the range of 3 to 5 wt %. and second step reduces the residual water content of the obtained protein particles below 0.5 wt %. The resulting protein particle suspensions are characterized by excellent chemical and physical stability, easy redispersibility and a favourable particle size distribution. The particle size distribution of the protein particle suspensions obtained by the method is ideal for injection purposes, avoiding clogging of a needle or cannula.

Accordingly, in a preferred embodiment, the step b) of removing water from the aqueous solution comprising the protein and the stabilizing agent is performed using a highly efficient drying method, preferably utilizing a drying method to result in a residual water content of the protein particles of less than 5 wt % or less than 3 wt %, or to result in a residual water content of the protein particles in the range of 3 to 5 wt % or in the range of 1 to 3 wt %, Suitable methods are known to the skilled person. Examples of such methods include lyophilization (i.e. freeze-drying) or spray drying.

Accordingly, in one embodiment, step b) comprises spray drying or lyophilization (or freeze-drying) of the aqueous solution comprising the protein and the stabilizing agent to obtain solid protein particles.

The second drying step may be performed with any, optionally other, suitable drying method, that allows to reduce the residual water content of the protein particles (further) to less than 0.5 wt % based on the total weight of the particle. A preferred method is vacuum drying.

In one embodiment, the suspension formulation may be obtained via a step b) comprising the spray drying of an aqueous solution comprising the protein and stabilizing agent, and optionally a further excipient (e.g. buffering agent such as histidine) to obtain the protein particles. In another embodiment, the suspension formulation may be obtained via a step b) comprising the lyophilization (i.e. freeze-drying) of an aqueous solution comprising the protein and stabilizing agent, and optionally a further excipient (e.g. buffering agent such as histidine) to obtain the protein particles.

In step b), spray drying may be conducted using, for example, but not limited to, a cyclone spray drier. The inlet/outlet temperature of the spray dryer used in the method should be such be at temperatures which do not affect the loss or degradation of protein. In one embodiment, the spray drying processing temperatures does not exceed 130° C. In another embodiment, the spray drying process does not exceed, i.e. is no more than 130° C. (inlet temperature) and no more than 70° C. (outlet temperature).

In a preferred embodiment, step c) above comprises vacuum-drying the particles of step b). In said step c), the vacuum drying may be conducted at an ambient temperature, or slightly higher than ambient temperature, for example between 15° C. to 40° C. in some embodiments, the vacuum drying may be conducted at temperatures of between 20 to 35° C., or 22 to 35° C., or 25-33° C., or 27 to 32° C. The vacuum drying is preferably performed at a reduced pressure, for example between 0.01 to 100 mbar. In other embodiments, the vacuum drying may be conducted at 0.01 to 10 mbar, 0.01 to 1 mbar, or at 0.01 mbar. In one embodiment, the vacuum-drying of step c), of the particles obtained from step b) may be conducted between 15-40° C., at a pressure of between 0.01-100 mbar. The duration of the vacuum drying in step b) may be at least 6 hours, or at least 12 hours or at least 24 hours. In one embodiment, step c) is conducted at a temperature between 15-40° C., at a pressure of about 0.01-100 mbar, for a period of at least 24 h. In an alternative embodiment, the vacuum drying step c) may be conducted no more than 24 hours. Step c) may be conducted so as to obtain protein particles with a residual water content of less than 0.5 wt % based on the total weight of the particle.

All compounds utilized in the method may be dried or free of water. Such the resulting suspension formulation is essentially free of water or substantially free of water. In some embodiments of the invention the residual water content of the suspension formulation is less than 0.5 mg/ml (or less than 0.05% (v/v), based on the total volume of the formulation.

In step d) the protein particles are suspended in a non-aqueous liquid vehicle comprising a semifluorinated alkene, a medium chain triglyceride (MCT), ethyl lactate, ethyl oleate or mixtures thereof.

Preferably, in step d) the protein particles are suspended in a non-aqueous liquid vehicle comprising a semifluorinated alkene. Semifluorinated alkanes are substantially non-toxic and are found to be well-tolerated by various types of human and animal tissue when administered topically or parenterally. In addition, they are chemically inert and are generally compatible with active and inactive ingredients in pharmaceutical formulations. Their typical densities range from 1.1 to 1.7 g/cm³, and their surface tension may be as low as 19 mN/m.

Semifluorinated alkanes are linear or branched alkanes where some of the hydrogen atoms are replaced by fluorine atoms. In one embodiment, the semifluorinated alkanes (which may be abbreviated as SFAs) described and used in the present disclosure comprise of one linear non-fluorinated hydrocarbon segment and of one linear perfluorinated hydrocarbon segment, preferably with the perfluorinated hydrocarbon segment attached to the non-fluorinated hydrocarbon segment.

In an embodiment, the semifluorinated alkanes have the chemical formula F(CF₂)ₙ(CH₂)ₘH, wherein n and m are integers defining the number of carbons in the perfluorinated hydrocarbon segment and non-fluorinated hydrocarbon segment respectively. In a further embodiment, the one or more semifluorinated alkanes is a semifluorinated alkane of formula F(CF2)n(CH2)m, wherein n is an integer selected from 4 to 6 and m is an integer selected from 2 to 10. In a further embodiment, the one or more semifluorinated alkanes is a semifluorinated alkane of formula F(CF₂)ₙ(CH₂)ₘ, wherein n is an integer selected from 4 to 6 and m is an integer selected from 4 to 8.

A nomenclature which is frequently used for semifluorinated alkanes designates a perfluorinated hydrocarbon segment as RF and a non-fluorinated segment as RH. Alternatively, the compounds may be referred to as FnHm and FnHm, respectively, wherein F means a perfluorinated hydrocarbon segment, H means a non-fluorinated segment, and n and m define the number of carbon atoms of the respective segment. For example, F3H3 is used for perfluoropropylpropane, F(CF₂)₃(CH₂)₃H. Moreover, this type of nomenclature is usually used for compounds having linear i.e. unbranched segments. Therefore, unless otherwise indicated, it should be assumed that F3H3 means 1-perfluoropropylpropane, rather than 2-perfluoropropylpropane, 1-perfluoroisopropylpropane or 2-perfluoroisopropylpropane.

Semifluorinated alkanes of the RFRH type are insoluble in water but also somewhat amphiphilic, with increasing lipophilicity correlating with an increasing size of the non-fluorinated segment. The semifluorinated alkanes used in the context of the present disclosure are preferably liquid semifluorinated alkanes.

In one embodiment of the present disclosure, the non-aqueous vehicle may comprise of one or more semifluorinated alkanes selected from the group consisting of F4H4, F4H5, F4H6, F4H8, F6H2, F6H4, F6H6, F6H8 and F6H10. or the non-aqueous vehicle may comprise of one or more semifluorinated alkanes selected from the group consisting of F4H4, F4H5, F4H6, F4H8, F6H4, F6H6, F6H8 and F8H8, or the non-aqueous vehicle may comprise of one or more semifluorinated alkanes selected from the group consisting of F4H5, F4H6, F4H8, F6H6 and F6H8 The chemical formula of these semifluorinated alkanes may be expressed, respectively as F(CF₂)₄(CH₂)₄H, F(CF₂)₄(CH₂)₅H, F(CF₂)₄(CH₂)₆H, F(CF₂)₄(CH₂)₈H, F(CF₂)₆(CH₂)₂H, F(CF₂)₆(CH₂)₄H, F(CF₂)₆(CH₂)₆H F(CF₂)₆(CH₂)₈H and F(CF₂)₆(CH₂)₁₀H. In another embodiment, the non-aqueous vehicle essentially consists of one or more semifluorinated alkanes selected from the group consisting of F4H4, F4H5, F4H6, F4H8, F6H4, F6H6 and F6H8.

In one embodiment, the suspension formulation according to the present disclosure comprises a protein particle such as defined herein suspended in a non-aqueous vehicle comprising, or essentially consisting of F6H8. In another embodiment, the suspension formulation according to the present disclosure comprises a protein particle as defined herein suspended in a non-aqueous vehicle comprising, or essentially consisting of F4H5. In another embodiment, the suspension formulation according to the present disclosure comprises a protein particle such as defined herein suspended in a non-aqueous vehicle selected from F4H5 and F6H8.

Optionally, the formulation may comprise more than one semifluorinated alkane. It may be useful to combine SFAs, for example, in order to achieve a particular target property such as a certain density or viscosity. If a mixture of semifluorinated alkanes is used, it is preferred that the mixture comprises at least one of F4H4, F4H5, F4H6, F4H8, F6H4, F6H6, F6H8, or comprises at least one of F4H4, F4H5, F4H6, F4H8, F6H2, F6H4, F6H6, F6H8, F6H10. In one embodiment, the non-aqueous vehicle may comprise at least two members selected from the group consisting of F4H4, F4H5, F4H6, F4H8, F6H4, F6H6 and F6H8, or may comprise at least two members selected from the group consisting of F4H4, F4H5, F4H6, F4H8, F6H2, F6H4, F6H6, F6H8 and F6H10

As used herein, the non-aqueous vehicle comprising a semifluorinated alkane comprises at least one or more semifluorinated alkanes. Said vehicle may optionally further comprise other vehicle or carrier compounds, or excipients, as such as further described herein. In one embodiment, the liquid vehicle comprises more than one semifluorinated alkane. In another embodiment, the liquid vehicle essentially consists of a semifluorinated alkane, or essentially consists a mixture of semifluorinated alkanes such as any one of the semifluorinated alkanes defined herein. In another embodiment, the suspension formulation comprises a non-aqueous vehicle consisting of one or more semifluorinated alkanes and optionally one or more pharmaceutically acceptable excipients, preferably excipients which are miscible, or soluble in the semifluorinated alkane or semifluorinated alkane mixture. In one embodiment, the non-aqueous vehicle comprises a semifluorinated alkane, or a mixtures of semifluorinated alkanes in an amount of at least 70 wt %, 75 wt %, 85 wt %, 90 wt %, 95 wt % or at least 99 wt % with respect to the total weight of the liquid vehicle. In a further embodiment, the non-aqueous vehicle essentially consists of 100 wt % of a semifluorinated alkane, or a mixture of semifluorinated alkane such as defined above.

In another embodiment, the method may comprise step e), homogenizing the suspension formulation. The homogenization may be conducted by any homogenization technique known in the art, e.g. using a high-shear homogenizer, or by ultrasound, which optionally may be conducted under cooling conditions (e.g. under ice-cooling conditions, such as around 0° C.).

In a preferred embodiment the method includes step e) and the homogenization is carried out using ultrasonication. In a further embodiment, the ultrasonication is performed below ambient temperature, preferably it is performed under cooling, such as under ice-cooling (in an ice bath).

In another embodiment, the method may comprise an optional step of selecting protein particles with a desired or predetermined particles size, with the particle size being defined by mean particle size diameter. Preferably, said selection of the protein particles with a desired or predetermined particle size may be carried out before suspending the protein particles in the non-aqueous vehicle. The selection of protein particles with a desired or predetermined particles size may be carried out by any method known to the skilled person; the selection step may include an additional milling step to generate or increase the number of particles with the desired or predetermined smaller particle size and/or may include a step of sorting out (i.e. by picking, sieving) the particles with the desired or predetermined particle size, to be suspended. The desired or predetermined particle size is defined by the application or medical use of the suspension formulation. For example, when utilized for injection purposes, such as subcutaneous, intramuscular or ocular injection, the predetermined particle size may be characterized by a distribution of at least 90% of the particles having a mean diameter of between 1 and 15 μm, or between 1 and 30 μm, or between 1 and 50 μm, or the predetermined particle size may be characterized by a mean diameter of less than 50 μm, less than 30 μm, less than 15 μm, between 1 and 15 μm, between 1 and 30 μm or between 1 and 50 μm, each as determined by laser diffraction. In a further embodiment, the aqueous solution comprises the protein and stabilizing agent, wherein the relative weight ratio of the protein to the stabilizer is between 1:1 to 7:3.

The method is suitable to obtain protein particle suspensions of all kinds of proteins. Said proteins may include naturally occurring and artificially generated proteins. In some embodiments the protein in the aqueous solution is selected from an antigen-binding polypeptide or protein, a vaccine and an enzyme. In some embodiments the protein is an antibody or fragment thereof.

In some embodiments the protein has a molecular mass of between 10 to 300 kDa.

The method is compatible with a wide range of stabilizing agents in the suspension. Examples of stabilizing agent include, but are not limited to, saccharides, polyols, amino acids, amines, surfactants, antioxidants, polymers, salts or combinations thereof. In some embodiments the stabilizing agent is selected from saccharides, polyols, amino acids, amines, surfactants, antioxidants, polymers, salts or combinations thereof. In some embodiments the stabilizing agent is a saccharide, preferably a saccharide selected from trehalose and sucrose.

The main advantage of the method is that the size distribution of the protein particles in the suspension is quite uniform and the particles are small. In one embodiment the method produces a suspension formulation comprising protein particles, wherein at least 90% of the protein particles have a mean diameter of between 1 and 30 μm, or between 1 and 50 μm as determined by laser diffraction.

The protein concentration in the suspension formulation may be adapted in step d) to the suitable needs. In some embodiments the protein is suspended in a sufficient amount of liquid vehicle that the protein concentration in the suspension formulations is between 2 and 350 mg/ml, between 2 and 250 mg/ml, or between 2 and 125 mg/ml. In some embodiments the total solid content of the suspension formulation is between 4 and 700 mg/ml, between 7 and 500 mg/ml, or between 4 and 250 mg/ml.

The protein particles may include further compounds aside from a protein and a stabilizing agent. In some embodiments the protein particles or the liquid vehicle may additionally comprise a surfactant. In a preferred embodiment, the suspension formulation obtained by the method is free of a surfactant.

In a related aspect, the present disclosure may also relate to a suspension formulation obtainable or obtained according to any one of method embodiments described above.

In a further aspect, the disclosure relates to a suspension formulation comprising a twice dried protein particle suspended in a non-aqueous vehicle, wherein the particle comprises a protein and a stabilizing agent, and wherein the residual water content of the suspended protein particle is less than 0.5 wt % based on total weight of the particle. Herein, the disclosure relates to a suspension formulation comprising a twice dried protein particle suspended in a non-aqueous vehicle, wherein the particle comprises a protein and a stabilizing agent, and wherein the residual water content of the suspended protein particle has been reduced (from an initial aqueous solution) by two consecutive drying steps to less than 0.5 wt % based on total weight of the particle.

Said suspension formulation may be obtained using a method as described above. Any particular embodiments of the suspension formulation described herein may be applied or realized in the method above.

In the context of the present invention “twice dried protein particle” refers to solid protein particles, obtained by drying a protein composition with two different methods. It is preferred that the two different drying methods are conducted consecutively. Preferably, the twice dried particles are obtained by first drying an aqueous solution comprising a protein and a stabilizing agent with a highly efficient drying method, such as spray-drying or lyophilization (i.e. freeze-drying) to obtain solid protein particles and a (consecutive) second drying step, such as vacuum drying. Herein, the first drying generates protein particles characterized by a residual water content in the range of 3-5 wt %, wherein the second drying step reduces the residual water content even further to less than 0.5 wt %, based on the total weight of the particles.

The formulation of protein or protein particles described herein is provided in the form of a suspension. A suspension may be defined as a type of a dispersion, i.e. a system having at least one continuous (or coherent) phase and at least one discontinuous (or inner) phase which is dispersed in the continuous phase. In a suspension, the dispersed phase is essentially in the solid state. In one embodiment of the present disclosure, the protein particles are insoluble in the continuous phase, wherein the continuous phase is comprised of a non-aqueous liquid vehicle, and are featured in the suspension formulation as the dispersed phase. In a preferred embodiment, the suspension formulations according to a present disclosure are liquid suspensions, at least at physiological temperature, meaning that the continuous phase is a liquid. Typically, the suspensions are also liquid at room temperature.

The non-aqueous vehicle as used and defined herein may form the continuous phase of the suspension formulation. The non-aqueous vehicle is preferably a liquid at room temperature. As understood herein, the term ‘non-aqueous’ in reference to a vehicle or any formulation component refers a vehicle or formulation component which is essentially free of water. In another embodiment, the non-aqueous vehicle is liquid, and also non-miscible with water. The term ‘a vehicle’ as used herein may refer to a vehicle consisting essentially of only a single component or compound which forms the continuous phase of the suspension formulation, or may refer to a vehicle comprising a combination of two or more components or compounds, which preferably are miscible and form a single continuous phase of the suspension formulation.

In one embodiment, suspension formulation comprises a protein particle as defined according to the present disclosure, suspended in a non-aqueous vehicle, wherein the non-aqueous vehicle comprises a semifluorinated alkane, a medium chain triglyceride (MCT), ethyl lactate, ethyl oleate or mixtures thereof. In alternative embodiment, the non-aqueous vehicle is selected from a group consisting of a semifluorinated alkane, a medium chain triglyceride (MCT), ethyl lactate, ethyl oleate and mixtures thereof.

In one embodiment, the non-aqueous vehicle comprises one or more semifluorinated alkanes.

As understood herein, the phrase ‘essentially consists of or’ essentially consisting of and the phrase ‘consists of or’ consisting of are considered to be interchangeable, and means that no further components are featured in the composition or formulation, other than those listed. If any other constituent or component, such as material-inherent impurities, are present in the composition or formulation, then these may be present only in negligible or trace amounts and confer no technical contribution, advantage or function in regards to the disclosed composition or formulation. The term ‘comprises’ or ‘comprising’, as used herein is in contrast to be construed in an open sense, where other features, for example composition components, other than those prefaced by the term may be present.

Moreover, the terms ‘about’, ‘substantially’ ‘essentially’ and the like in connection with an attribute or value such as concentration or amount as used herein includes the exact attribute or precise value, as well as any attribute, or value typically considered to fall within a normal range or accepted variability associated in the technical field and the methods of measurement or determination of said attribute or value.

In one embodiment, the suspension formulation comprises a liquid vehicle comprising one or more semifluorinated alkanes, wherein the semifluorinated alkane is present in an amount of at least 70 wt %, 85 wt %, 90 wt %, or at least 95 wt % by total weight of the formulation (wt %). In another embodiment, the semifluorinated alkane may be present in an amount of about 85 to 99% by weight of the formulation.

The term ‘protein particles’ as understood herein refer to solid particles comprising of a protein that are substantially non-soluble in the non-aqueous vehicle of the suspension formulation, and which are thus are featured as particles dispersed or suspended in the continuous phase formed by the vehicle. The particle as defined herein may comprise of a protein, a stabilizing agent, and optionally one or more additional excipients which are combined together in accordance with the process of preparing the particle, such as further defined herein, together to form a unit solid phase which may be dispersed or suspended in a liquid, non-aqueous vehicle.

As used herein, the singular forms ‘a’, or ‘an’, or ‘the’ does not exclude a plurality, i.e. these terms may be understood, in addition to the singular form and meaning, as including also the plural form or plurality unless context clearly indicates, requires, or implies otherwise. In other words, references to singular characteristics or limitations of the present disclosure may include the corresponding plural characteristic or limitation, and vice versa, unless explicitly specified otherwise or clearly implied to the contrary by the context in which the reference is made. As an example, the use of the term ‘a’, or ‘an’ or ‘the’ such as in reference to ‘a’ protein particle will have the same meaning as ‘at least one’, or ‘one or more’ protein particles, unless defined otherwise.

The term ‘protein’ as used herein may be interchangeable with the term ‘polypeptide’. A polypeptide may also be referred to as a protein, and vice versa. Typically, the term “polypeptide” only refers to a single polymer chain, whereas the expression “protein” may also refer to two or more polypeptide chains that are linked to each other by non-covalent bonds. Polypeptides and proteins in general represent polymers of amino acid units that linked to each other by peptide bonds. Since the size boundaries that are often used to differentiate between polypeptides and proteins are somewhat arbitrary, the two expressions for these molecules should—within the context of the present disclosure—not be understood as mutually exclusive.

In one embodiment of the present disclosure, the protein particle suspended in a non-aqueous vehicle comprises a protein having a molecular mass of between 3 to 200 kDA or between 10 to 200 kDa. In another embodiment, the protein has a molecular mass of between 50 to 200 kDa, or between 50 to 150 kDa, or between 100 to 200 kDa, or between 50 to 100 kDa, or between 3 to 50 kDa or between 3 to 25 kDa.

In a further embodiment of the present disclosure, the protein particle suspended in a non-aqueous vehicle comprises a protein, which is a polypeptide comprising about 25 to 200 amino acids, preferably comprising about 25 to 100 amino acids, or more preferably 25 to 50 amino acids.

In one embodiment, the protein particle may comprise an antigen-binding polypeptide or protein. As used within the context of the present disclosure, the term antigen-binding polypeptides or proteins refer to full-length and whole antibodies, also known as immunoglobulins, in their monomer, or polymeric forms as well as any fragments, chains, domains or any modifications derived from a full-length antibody capable of specifically binding to an antigen. The antigen-binding polypeptides or proteins may belong to any of the IgG, IgA, IgD, IgE, or IgM immunoglobulin isotypes or classes. In one embodiment, the protein as used herein may be an immunoglobulin G (IgG) antibody, i.e. an antibody, antibody fragment, or protein comprising an antibody fragment derived from immunoglobulin G or any one of its five classes (e.g. IgG1, IgG2, IgG3, IgG4).

In one embodiment, the protein particle may comprise an enzyme, e.g. a lysozyme. Lysozymes are a glycoside hydrolase enzymes which hydrolyse glycosidic bonds such as found in peptidoglycans. Lysozymes may function as an antimicrobial, in particular against gram-positive bacteria and bacteria where peptidoglycan is prominently featured in bacterial cell wall. Lysozyme (type c) has a molecular mass of about 14.3 kDa. In another embodiment, the enzyme may be an enzyme that is deficient or produced at low levels in a subject in need thereof.

In one embodiment, the protein particle may comprise a protein vaccine, such as a purified or recombinant proteinaceous antigen from a pathogen, such as a bacterium or virus.

In another embodiment, the protein particle may comprise a protein selected from an enzyme and an antigen-binding polypeptide or protein, such as an antibody or an immunoglobulin (for example an immunoglobulin G antibody, preferably a human or humanized IgG1), or an antigen-binding antibody fragment, or a fusion protein comprising an antibody fragment, or an antibody-drug conjugate. In yet a further embodiment, the protein is selected from the group consisting of a lysozyme and an antibody or an immunoglobulin.

In a particular embodiment, the protein particle comprises an antibody. The term ‘antibody’ may refer to a full-length antibody, as well as any fragments, chains, domains or any modifications derived from a full-length antibody capable of specifically binding to an antigen.

A full-length antibody is a Y-shaped glycoprotein comprising of a general structure with an Fc (fragment crystallisable) domain and a Fab (fragment antigen binding) domain. These are structurally composed from two heavy (H) chains and two light (L) chain polypeptide structures interlinked via disulphide bonds to form the Y-shaped structure. Each type of chain comprises a variable region (V) and a constant region (C); the heavy chain comprises a variable chain region (V_{H}) and various constant regions (e.g. CH₁, CH₂, etc.) and the light chain comprises a variable chain region (V_{L}) and a constant region (CO. The V regions may be further characterized into further sub-domains/regions, i.e. framework (FR) regions comprising more conserved amino acid residues and the hypervariable (HV) or complementarity determining regions (CDR) which comprise of regions of increased variability in terms of amino acid residues. The variable regions of the chains determine the binding specificity of the antibody and form the antigen-binding Fab domains of an antibody.

Antibody fragments as used herein may include any region, chain, domain of an antibody, or any constructs or conjugates thereof that can interact and bind specifically to an antigen, and may be monovalent, bivalent, or even multivalent with respect to binding capability. Such antibody fragments may be produced from methods known in the art, for example, dissection (e.g. by proteolysis) of a full-length native antibody, from protein synthesis, genetic engineering/DNA recombinant processes, chemical cross-linking or any combinations thereof. Antibody fragments are commonly derived from the combination of various domains or regions featured in variable V region of a full-length antibody. In one embodiment, the protein particles may comprise an antibody fragment, for example wherein the antibody fragment is a fragment antigen-binding (Fab), a single-chain variable fragment (scFv), a single-domain antibody, a minibody, or a diabody. The fragment may be a single-chain variable fragment (scFv) such as those comprising of heavy (V_{H}) and light (V_{L}) chain variable domains joined by a linker or a complexed multimeric/multivalent constructs thereof, for example, diabodies (bivalent dimer), triabodies (trivalent trimer), or tetrabodies (tetravalent tetramer). Multimeric antibody fragments may also be multispecific, for example, a bispecific diabody may comprise of two fragments each with specificity for a different antigen. Further preferred antibody fragments include single domain antibodies (daBs) such as those comprising a single V_{H} or V_{L} domain capable of specifically binding to an antigen. Antibody fragments also within the scope of the disclosure may include scFv-CH dimer constructs such as a minibody.

In one embodiment, the protein comprised in the particle according to the disclosure is a monoclonal antibody (mAb). A monoclonal antibody refers to an antibody obtained from a homogenous population of antibodies that are specific towards a single epitope or binding site on an antigen. Monoclonal antibodies may be produced using antibody engineering techniques known in the art, such as via hybridoma or recombinant DNA methods. In one embodiment, the protein particle according to the present disclosure comprises a recombinant monoclonal antibody. In further embodiment, the protein as used herein may be selected from a chimeric, humanized or human monoclonal antibody. Chimeric monoclonal antibodies, for example, refer to hybrid monoclonal antibodies comprising domains or regions of the heavy or light chains derived from antibody sequences from more than one species, for example from murine and human antibody sequences. Humanized monoclonal antibodies may refer to antibodies which are predominantly structurally derived from human antibody sequences, generally with a contribution of at least 85-95% human-derived sequences, whereas the term ‘human’ refers to those derived solely from human germline antibody sequences. In one embodiment, the protein according to the present disclosure is a recombinant humanized or human monoclonal antibody, preferably an immunoglobulin G antibody or immunoglobulin G1 antibody.

In another embodiment, the protein particle may comprise a fusion protein. Fusion proteins as defined herein are comprise of at least one antibody fragment capable of specifically binding to an antigen, fused to at least one other bioactive protein or polypeptide, or fragment thereof. In one embodiment, the protein particle as defined herein may comprise of an Fc-fusion protein, a protein comprised of an immunoglobulin Fc domain covalently linked to at least another peptide or peptide fragment.

Antibody-drug conjugates comprising an antibody or antibody fragment covalently conjugated or linked to a drug molecule (for example a small molecule drug, or a radiolabelled component) are also within the definition of antigen-binding polypeptides or proteins as used herein.

In one specific embodiment, the protein is selected from bevacizumab, aflibercept and ziv-aflibercept. Aflibercept (commercial name, EYLEA) is an recombinant Fc-fusion polypeptide carrying extracellular domains of VEGF receptors (VEGF 1 and 2), being used as decoy receptor to neutralize VEGF. Aflibercept may be used for treating patients suffering from Neovascular (Wet) Age-related Macular Degeneration (AMD), Macular Edema following Retinal Vein Occlusion (RVO), Diabetic Macular Edema (DME) and Diabetic Retinopathy (DR). Bevacizumab is a recombinant humanized monoclonal antibody that blocks angiogenesis by inhibiting vascular endothelial growth factor A (VEGF-A). Bevacizumab is a full-length IgG1κ isotype antibody composed of two identical light chains and two heavy chains with a total molecular weight of 149 kDa. The two heavy chains are covalently coupled to each other through two inter-chain disulphide bonds, which is consistent with the structure of a human IgG1.

As defined herein, the protein particle according to the present disclosure comprises a protein and a stabilizing agent. A ‘stabilizing agent’ as referred to herein may be any excipient, or a combination of two or more excipients which stabilizes a protein, protein particle or suspension formulation as such. The stabilizing agent may provide a protective effect against mechanical, physical, chemical stress, or a combination thereof during manufacturing processes, or during storage. For example, the stabilizing agent may be useful for preventing instability of the protein during the spray-drying and exposure to temperature extremes, such as elevated temperatures. Examples of stabilizing agent include, but are not limited to, saccharides, polyols, amino acids, amines, surfactants, antioxidants, polymers, salts or combinations thereof.

In one embodiment, the stabilizing agent is a saccharide or a sugar. The saccharide or sugar may be a monosaccharide, disaccharide, trisaccharide, or optionally a oligosaccharide or a polysaccharide. Examples of saccharides which may function as a stabilizing agent include glucose, fructose, galactose, sucrose, maltose, trehalose, maltose, lactulose, lactose, or cyclodextrins. In one preferred embodiment, the stabilizing agent is selected from trehalose, sucrose, or a combination thereof. The saccharide or sugar featured or used in the manufacture of the protein particle is in one embodiment, amorphous.

In another embodiment, the stabilizing agent is a polyol. Examples of polyol include sugar alcohols such as, but not limited to, glycerol, arabitol, erythritol, mannitol, sorbitol, xylitol, maltitol, lactitol. In yet another embodiment, the stabilizing agent is a saccharide, a polyol, a polysorbate or a combination thereof.

As used herein, the term ‘excipient’ refers to any pharmaceutically acceptable agent or combination of agents which may be added to a pharmaceutical formulation or a composition in an amount which provides or adjusts a particular property or characteristic of said formulation or composition. Examples of excipients include surfactants, chelating agents, buffer agents, pH-modifying agents such as inorganic salts or organic salts, antioxidants or reducing agents, bulking agents, organic co-solvents, as well as stabilizing agents such as described above. An excipient may provide or have more than one function in a formulation. The excipients as used herein are preferably acceptable for pharmaceutical use, meaning that the compound or mixtures used as excipients are non-toxic and acceptable for human pharmaceutical use. Preferably, the excipients are suitable for parenteral, topical, dermatological or ophthalmic use.

Examples of surfactants are non-ionic surfactants and ionic surfactants. Examples of surfactants which may be used in the context of the present disclosure include, but are not limited to polysorbates and poloxamers. Poloxamers are triblock copolymers of polyoxyethylene and polyoxypropylene; examples include poloxamer P188. Polysorbates are pegylated sorbitan fatty acid esters; examples which may be useful according to the present disclosure include, but are not limited to polysorbate 20 (polyoxyethylene sorbitan monolaurate), polysorbate 40 (polyoxyethylene sorbitan monopalmitate), polysorbate 60 (polyoxyethylene monostearate) and polysorbate 80 (polyoxyethylene monooleate). In one embodiment, the suspension formulation comprises a surfactant. In a further embodiment, the suspension formulation is free of a surfactant.

Examples of chelating agents which may be used in the context of the present disclosure are EDTA, citrate. Examples of antioxidants include alpha-tocopherols, butylated hydroxytoluene, ascorbic acid, cysteine, methionine. Examples of inorganic salts includes calcium, magnesium, zinc, or sodium salts, such as carbonates (e.g. calcium carbonate), hydroxides, phosphates, hydrogen phosphates, acetates, or chlorides (e.g. NaCl). Examples of amino acids include arginine, histidine, glycine, glutamate, asparagine, and the like. Examples of polymers include polyvinylpyrrolidone, celluloses, polysaccharides

In one embodiment, the formulation includes a buffering agent which controls changes in pH, for example, a buffer which control pH in the range of 5.0 to 7.0, or about pH 6.0. Examples of buffering agents include but are not limited to histidine, glycine, acetate, succinate, gluconate, citrate, tris, glutamate, and phosphate.

In one embodiment, the protein particles may comprise a protein, stabilizing agent and optionally, one or more further excipient. In said embodiments, the stabilizing agent is different from the one or more further excipients. In one embodiment, the protein particle may comprise, or consist, further to the protein and stabilizing agent, a buffering agent. In one said embodiment, the buffering agent may be histidine. In another embodiment, the protein particle may comprise, or may consist further to the protein and a stabilizing agent, a buffering agent (e.g. histidine) and a surfactant (e.g. polysorbate). In other embodiments, the protein particle may essentially consist of the protein and the stabilizing agent, and optionally one or more further excipients.

The relative ratio, based on weight, of the protein to stabilizing agent in the protein particles may be in the range of 1:1 to 7:3. In other embodiments, the relative weight ratio of the protein to stabilizing agent may be about 50:50, 55:45, 60:40, 65:35, 70:30; or may be in the range of 55:45 to 70:30, 60:40 to 70:30, or 65:35 to 70:30. In one specific embodiment, the protein particle comprises an antibody or an enzyme, and the stabilizing agent is a saccharide (e.g. trehalose or sucrose), wherein the relative weight ratio of these in the protein particle is between 1:1 to 7:3.

According to the present disclosure, the residual water content of the suspended protein particles is less than 0.5 wt %, based on the total weight of the particle. As understood herein, the term “water content’ or ‘residual water content’ refers to the amount of water present in a composition (e.g. protein particles), or the amount of water remaining in a composition, such as after processing or manufacturing thereof, which may comprise a step of removal of water.

In one embodiment, the residual water content of the suspended particles based on the total weight of the particle is less than 0.5 wt. %. In further embodiments, the suspended protein particles may have a residual water content of equal, or less than 2.0, 1.0, 0.8, 0.6, 0.5, 0.4, 0.3, 0.2, 0.1, or 0.05 wt %, based on the total weight of the protein particle. In yet further embodiments, the residual water content of the suspended protein particles as defined herein may be in the range of 0.05 to 0.5 wt %, or 0.05 to 0.2 wt %, 0.1-0.5 wt % 0.1-0.2 wt %, based on the total weight of the particles.

Formulations in the form of a suspension, including protein particle suspensions or dispersions need to be physically stable in order to be suitable for use in therapeutic applications. After storage, or standing over a period of time the dispersed particle phase may separate from the liquid continuous phase of a suspension such as by flotation or sedimentation of particles. The physical stability of a suspension may be determined, for example by the rate of sedimentation/flotation or the ease of redispersion of the particles.

For dosing accuracy and reproducibility, especially for injectable or parenteral suspensions where the ease of injection may be affected, the particles in suspension formulations preferably should maintain a consistent particle size distribution and be easily redispersible. Moreover, the suspension formulations should remain homogenously dispersed and flotation or sedimentation should only occur slowly. As used herein, the term ‘redispersible’ which may be used interchangeably with the term ‘resuspendable’ and the like, refers to the ability of a suspension to substantially reform or revert back to its initial or intended suspension profile, after settling or phase separation.

Suspension formulations which are less suitable and which are less stable tend to be poorly redispersible, whereby agglomerates or cakes of protein particles which may be formed cannot be as easily redispersed, where phase separation of the dispersed particles occurs rapidly, and where particle size distribution changes over a period of time, for instance due to occurrence of particle aggregation. The formation of dense and poorly redispersible protein particle agglomerates may make precise dosing challenging, or in some cases, impossible, if the size of the agglomerates may lead to the clogging of fine-gauged needles typically used for, e.g. subcutaneous injections.

It has been unexpectedly found that further improvements in the physical stability of suspension formulations comprising protein particles dispersed in a non-aqueous liquid vehicle, such as a semifluorinated alkane, may be achieved when the protein particles have a water content of at least less 0.5 wt %, or within the ranges as defined above. As described herein, it has been found that these suspension formulations, which for example, may be obtainable by a process comprising a step of spray drying, followed by a subsequent step of vacuum drying, provides suspensions which may have improved characteristics such as redispersibility, physical stability, and injectability and/or syringeability, also over prolonged periods of time compared to suspensions prepared from protein particles prepared by spray-drying only which typically have a residual water content in the range of 3 to 5 wt %.

In particular, it was found that the dispersion properties of the suspensions when initially prepared were superior to suspensions prepared from same protein particles but comprising a higher residual water content. It was also found that the physical stability of said suspensions, as determined, for example, by particle size growth and redispersibility as well as injectability/syringeability (see Examples) may also be maintained over a prolonged storage (up to 4, 6 or 12 months, for example) even at elevated (stress level) temperatures of up to 40° C.

These suspension formulations may accordingly be useful as formulation and delivery vehicles for therapeutic proteins and polypeptides and/or as a storage or transport medium for said proteins and polypeptides.

Alternatively, water content may also be determined for the suspension formulation as such. Preferably, the total residual water content of the suspension formulation may be less than 1.0 mg/ml, or less than 0.5 mg/ml, based on the total volume of the formulation. In one embodiment, the total residual water content of the suspension formulation may be less than 1.0, 0.9, 0.8, 0.7, 0.6, 0.5, 0.4, 0.35, 0.3, 0.25, 0.2, 0.15, 0.1, 0.05 mg/ml, based on the total volume of the formulation. In other embodiments, the total residual water content of the suspension form may be in the range of 0.05 mg/ml to 1.0 mg/ml, or 0.005 mg/ml to 0.5 mg/ml, based on the total volume of the formulation. Also preferred, the total residual water content of the suspension formulation may be less than 0.1% (v/v), or less than 0.05% (v/v), based on the total volume of the formulation. In one embodiment, the total residual water content of the suspension formulation may be less than 0.1, 0.09, 0.08, 0.07, 0.06, 0.05, 0.04, 0.03, 0.02, 0.01% (v/v), based on the total volume of the formulation. In other embodiments, the total residual water content of the suspension form may be in the range of 0.001% (v/v) to 0.1% (v/v), or 0.001% (v/v) to 0.01% (v/v), based on the total volume of the formulation.

In a preferred embodiment, the total solid content suspension formulation the is up to 30 mg/ml and the total residual water content of the suspension formulation is less than 0.15 mg/ml (less than 0.015% (v/v)) or less than 0.03 mg/ml (less than 0.003% (v/v))) based on the total volume of the formulation. In a further preferred embodiment, the total solid content suspension formulation the is up to 50 mg/ml and the total residual water content of the suspension formulation is less than 0.25 mg/ml (less than 0.025% (v/v)) or less than 0.05 mg/ml (less than 0.005% (v/v))) based on the total volume of the formulation. In a still further preferred embodiment, the total solid content suspension formulation the is up to 100 mg/ml and the total residual water content of the suspension formulation is less than 0.5 mg/ml (less than 0.05% (v/v)) or less than 0.1 mg/ml (less than 0.01% (v/v))) based on the total volume of the formulation, In a still further preferred embodiment, the total solid content suspension formulation the is up to 300 mg/ml and the total residual water content of the suspension formulation is less than 1.5 mg/ml (less than 0.15% (v/v)) or less than 0.3 mg/ml (less than 0.03% (v/v))) based on the total volume of the formulation, The residual water content of the suspension formulation, protein particle or other components of the formulation may be determined by conventional techniques and analysis methods known in the art, for example by Karl Fischer analysis, loss on drying or thermogravimetric analysis.

The protein concentration in the suspension formulations according to the present disclosure may be between 2 and 350 mg/ml, or between 25 and 350 mg/ml. In other embodiments, the concentration of protein may be up to 280 mg/ml, up to 210 mg/ml, up to 140 mg/mL, up to 70 mg/mL, or up to 350 mg/ml. In further embodiments, the concentration of protein in the suspension formulation may be between 2 to 280 mg/ml, 5 to 280 mg/ml, 25 to 280 mg/mL; 25 to 210 mg/mL, 25 to 140 mg/mL, 70 to 210 mg/mL, 70 to 280 mg/mL, 140 to 280 mg/mL, or 210 to 280 mg/mL, 5 to 50 mg/ml.

In further embodiments, the suspension formulation may have a total solid content (TSC) of between 7 and 500 mg/ml, or between 50 and 500 mg/ml. As understood herein, total solid content may refer to the amount of solids (mass per volume) retained after removal of liquid phase of the formulation. In other embodiments, the total solids content of the suspension formulation may be up to 500 mg/ml. In further embodiments, the total solids content of the suspension formulation may be up to 400 mg/mL, 350 mg/mL, 300 mg/ml, 200 mg/ml or 100 mg/l In yet further embodiments, the total solids content may be between 7 to 450 mg/ml, 25 to 450 mg/ml, 50 to 400 mg/mL, 50 to 300 mg/mL, 100 to 300 mg/mL, 100 to 200 mg/mL, or between 200 to 300 mg/ml.

In one embodiment, the suspension formulation according to any one of the other embodiments or combination of embodiments described herein, may comprise between 50 to 70% of protein in respect to the total solid content (TSC) of the formulation. In other embodiments, the amount of protein with respect to the total solid content of the suspension formulation may be about 50%, 55%, 60%, 65%, or 70%. In another embodiment, the amount of protein with respect to the total solid content of the suspension formulation may be between 50 to 60%, 55% to 65%, or 60 to 70%).

The protein particles according to the present disclosure preferably have a mean diameter of less than 30 μm or less than 50 μm, as determined by laser diffraction. In further embodiments, the mean diameter of the protein particles may be less than 20 μm, 15 μm, 10 μm, or less than 5 μm, as determined using laser diffraction. Optionally, the particle size distribution base by which particle size of the suspension formulation may be determined using laser diffraction may be volume, or in other words, the ‘mean diameter’ may refer to volume mean diameter. In an optional embodiment, the protein particles may have a volume mean diameter of less than 50 μm, less than 30 μm, less than 20 μm, 15 μm, 10 μm, or less than 5 μm.

In further embodiments, the suspension formulations according to the present disclosure may comprise of protein particles, wherein at least 90% of the protein particles have a mean diameter of between 1 to 30 μm, or of between 1 to 50 μm, as determined by laser diffraction. Optionally, the particle size distribution base on which particle size of the suspension formulation is determined by laser diffraction may be volume, or in other words, ‘mean diameter’ may refer to a volume mean diameter. Optionally, at least 90% of the protein particles of the suspension formulation may have a volume mean diameter of less than 50 μm, less than 30 μm, less than 20 μm, less than 15 μm, less than 10 μm, or less than 5 μm.

The suspension formulation according to the present disclosure may, in one embodiment comprise of a protein particle consisting essentially of a protein, stabilizing agent, and optionally one or more excipients. In a further embodiment, the suspension formulation may consist essentially of a protein particle suspended in a non-aqueous vehicle, wherein said protein particle consists of a protein and stabilizing agent, and optionally one or more excipients.

In one embodiment, the suspension formulation may further comprise one or more excipients, such as defined above, for example a surfactant such as polysorbate 80 or polysorbate 20. In another embodiment, the suspension formulation according to the present disclosure does not comprise, or is free of a surfactant and/or a preservative. A preservative may be any excipient which is added as an antimicrobial, to prevent microbial contamination and growth in the formulation. An example of a preservative is benzalkonium chloride, 1,3-butandiol; phenol, benzyl alcohol.

In a further embodiment, the present disclosure relates to a suspension formulation comprising a protein particle suspended in a non aqueous vehicle comprising, or essentially consisting of a semifluorinated alkane, wherein: the relative weight ratio of the protein to stabilizer agent in the protein particle is between 1:1 to 7:3, the residual water content of the protein particle is less than 0.5 wt %; preferably less than 0.3 wt % relative to the total weight of the protein particle; and wherein the total solid content of the formulation is no more than about 300 mg/ml.

In said embodiment, the semifluorinated alkane is preferably selected from F4H5 or F6H8. In one embodiment, the total solid content of said formulation is 300 mg/ml. In an alternative embodiment, the total solid content of the formulation is no more than about 100 mg/ml. In further aspects of said embodiment, the residual water content of the formulation may be less than 0.4 wt %, or less than 0.25 wt %, based on the total weight of the formulation. The protein particle according to any one of these embodiments is preferably a spray-dried protein particle; more preferably a spray-dried and a vacuum dried protein particle.

The suspension formulation may, in another embodiment comprise of a protein particle suspended in a non-aqueous vehicle, wherein the non-aqueous vehicle essentially consists of a semifluorinated alkane selected from F4H5 or F6H8 (or a mixture thereof), and optionally one or more excipients, preferably wherein the one or more excipients are solubilized or soluble in F4H5 or F6H8; wherein the protein particle is a spray-dried (and vacuum-dried) particle comprising a protein, a stabilizer agent, and optionally one or more further excipients (e.g. a buffering agent, such as histidine); wherein the protein is a monoclonal antibody, or is selected from the group consisting of a lysozyme, an immunoglobulin, aflibercept, ziv-aflibercept, or bevacizumab; and wherein the stabilizer agent is a saccharide, preferably selected from sucrose and trehalose, and/or a polyol; wherein the relative weight ratio of the protein to stabilizer agent in the protein particle is between 1:1 to 7:3; wherein the total solid content of the formulation is no more than about 300 mg/mL, and wherein the residual water content of the protein particle is less than 0.5 wt %, preferably less than 0.3 wt % relative to the total weight of the protein particle.

The suspension formulation according to the present disclosure may administered by injection or parenteral administration. In one embodiment, the suspension may be withdrawn (aspirated) into a syringe as well as injected through a fine-gauge needle (e.g. 27G or 23 G needle). In one embodiment, the suspension formulation may be injected with an injection glide force of less than 35 N (Newton). In a further embodiment, the formulation may be injected with a glide force of less than 15 N. In yet further embodiments, the injection glide force of the formulation may be less than 25 N, 20 N, 15 N, or less than 10 N; or between 1 to 10 N, or between 5 to 15 N. Preferably, the injection glide force of the suspension formulation does not substantially change over a period of storage of at least up to 12 months. In a further embodiment, the injection glide force required for administering the suspension formulation may be less than 35 N, 25 N, 20 N, 15 N, or less than 10 N; between 1 to 35 N, or between 5 to 35 N after storage of the suspension formulation at 40° C. for at least up to 1, 2, 3, 4, 5, 6, 7, 8, 9, 10, 11 or 12 months.

In one specific embodiment, the suspension formulation comprises a non-aqueous vehicle comprising or essentially consisting of a semifluorinated alkane, preferably F4H5, or F6H8, wherein the injection glide force is less than 15 N, or less than 10 N, or between 1 to 15 N, or between 5 to 15 N. Preferably, the injection glide force of the suspension formulation does not substantially change over a period of storage of at least up to 12 months. In a further embodiment, said formulation may have an injection glide force of less than 15 N, or less than 14, 13, 12, 11, or 10 N; or between 1 to 15 N after storage at 40° C. for at least up to 1, 2, 3, 4, 5, 6, 7, 8, 9, 10, 11 or 12 months.

In another embodiment, the suspension formulation comprises a non-aqueous vehicle comprising or essentially consisting of an ethyl oleate or ethyl lactate, wherein the injection glide force is less than 20 N. Preferably, the injection glide force of said suspension formulation does not substantially change over a period of storage of at least up to 12 months. In a further embodiment, said formulation may have an injection glide force of less than 20 N, or between 5 to 20 N after storage at 40° C. for at least up to 1, 2, 3, 4, 5, 6, 7, 8, 9, 10, 11 or 12 months.

As understood herein, the injection glide force refers to the maximal force required for injection of the formulation through a needle and syringe. In any of these above embodiments, the injection glide force may be applicable for achieving a flow rate of 0.1 mL/s and injection with a 1-mL syringe and a 27G needle (inner diameter of ca. 210 μm), of a type corresponding or analogous to syringe and needles exemplified herein.

The suspension formulation according to the present disclosure preferably has a viscosity of 5 to 40 mPa s, as measured by rotational viscometry at 25° C. In some embodiments, the suspension formulation according to the present disclosure may have a viscosity of 10 to 30, 10 to 25, 10 to 20, 15 to 30, 15 to 25, or 15 to 30 mPa s as determined by rotational viscometry at 25° C. In other embodiments the suspension formulations may have a viscosity of about 10, 15, 20 25, 30 mPa s; or less than 40, 35, 30, 25, 20, 15, 10 mPa s, as measured by rotational viscometry at 25° C.

The particles of the suspension formulation according to the present disclosure, upon sedimentation or flotation may be re-dispersed, such as by rotation (e.g. using a vertical rotator), shaking by hand, or by shaking at a frequency of up to 15 Hz. In one embodiment, the suspension formulations as described herein may be redispersed by shaking at a frequency of up to 5 Hz, or 10 Hz or between 2 to 15 Hz, 2 to 10 Hz, 2 to 5 Hz, 5 to 15 Hz, 5 to 10 Hz). The shaking may be conducted using a device in the art such as described herein for redispersion of a suspension.

In one embodiment, the suspension formulation may be redispersible after storage at room temperature for at least 1 month. In further embodiments, the suspension formulation may be redispersible for at least 1, 3, 6, or 12 months. As used herein, the term ‘redispersible’ which may be used interchangeably with the term ‘resuspendable’, refers to the ability of a suspension to substantially reform or revert back to its initial, or intended suspension profile, after settling or phase separation, e.g. during period of storage. In a further embodiment, the formulation according to the present disclosure may be redispersible after storage at up to 40° C. for at least 1, 3, 6, or 12 months.

In another embodiment, the suspension formulation may be redispersed or resuspended in less than 1000 s and retains at least 70, 80 or 90% of its original particle size distribution. Preferably, the suspension formulation may be redispersed or resuspended in less than 1000 s and retains at least 70, 80 or 90% of its original d90 particle size distribution The time period refers to the time required for redispersion of a settled or phase-separated suspension, such as by a mechanical or physical means such as described herein (e.g. shaking by hand, or using a shaker). In other embodiments, the re-suspension of the suspension formulations according to the present disclosure may be conducted in less than 800, or 900 seconds, or within a period of 2 to 800, or 2 to 900, or 2 to 1000 seconds. In yet a further embodiment, the suspension formulation comprises a non-aqueous vehicle, wherein the non-aqueous vehicle is a semifluorinated alkane, and wherein the suspension formulation is redispersible in less than 50 s and retains at least 70%, 80% or at least 90% of its original particle size distribution. In a further embodiment, said suspension formulation may be redispersible in less than 20 s, or less than 30 seconds; or within 2 to 50 s, 2 to 30 s, or within 2 to 20 s. In yet another embodiment, said formulation may be redispersible within 30 s at a frequency of 5 Hz, or within 30 s by manual shaking to retain at least 70%, or 80%, or 90% of its original particle size distribution. In yet a further embodiment, the suspension formulation comprises a non-aqueous vehicle, wherein the non-aqueous vehicle is a medium-chain triglyceride (MCT), ethyl oleate or ethyl lactate, and wherein the formulation may be redispersed in less than 1000 seconds (s) to retain at least 70%, 80%, or 90% of its original particle size distribution. In a more specific embodiment, the non-aqueous vehicle may be a MCT, wherein the suspension formulation is resdispersible in less than 800 s, or less than 900 s; or within 200 to 1000 s, 200 to 900 s, 200 to 800 s, 300 to 1000 s, 300 to 900 s, or 300-800 s.

In one embodiment, the protein particle is a spray-dried or a lyophilized protein particle. The term ‘spray-dried’ as used herein refers to a protein particle which has been prepared using a spray-drying process comprising spray-drying an aqueous solution comprising a protein and a stabilizing agent, and optionally, one or more further excipients. The term ‘lyophilized’ refers to a protein particle which has been prepared by lyophilizing i.e. freeze-drying an aqueous solution comprising a protein and a stabilizing agent, and optionally one or more further excipients. In a preferred embodiment, the protein particles suspended in a non-aqueous vehicle as described herein are spray-dried protein particles. In a further embodiment, the protein particles suspended in a non-aqueous vehicle as described herein are spray-dried and additionally dried protein particles, namely protein particles obtained from spray-drying that were subsequently subjected to an additional drying step, such as an additional vacuum-drying step. In yet another embodiment, the protein particles may be lyophilized and additionally, dried protein particles, namely protein particles obtained from lyophilization that were subsequently subjected to an additional drying step, such as an additional vacuum-drying step. As understood herein, the term vacuum-dried refers to a protein particle which has undergone a vacuum drying process, which may be distinguished from lyophilization in that the vacuum drying is not conducted under cryogenic conditions such as performed in the art for lyophilization.

Freeze-drying (lyophilization) is a typical and often preferred method for the removal of water from protein particles, as the process of spray drying requires exposure of the protein particles to elevated temperatures, and as such may be associated with loss of protein or protein activity. It was observed, however that the protein particles according to the present disclosure prepared using spray drying and in combination with a step of vacuum drying conducted at about ambient or higher temperatures (for example in the range of 15-40° C.), and not under water sublimation conditions such as in lyophilization not only provided physically stable suspensions in non-aqueous vehicles of protein particles with a homogenous particle size distribution amenable for administration by injection, but where protein activity is also retained (see Example 2).

In a further aspect, the present disclosure relates to the use of the suspension formulation as described herein for therapeutic and/or diagnostic applications. The suspension formulation may be used for treating, for example diseases or conditions affecting the skin, eye, ear, nose, or lung in a subject in need thereof. As understood herein, ‘subject’ may refer to a human subject, and may also be used synonymously with the term ‘patient’. Said subject, or patient may be suffering or diagnosed with a disease or condition and requiring treatment or amelioration, improvement, control, control of progression, prevention of occurrence, etc of said disease or condition, or symptom(s) of said condition or disease. Optionally, the subject may be also be a veterinary subject.

In one embodiment, the suspension formulations may be used for the treatment of an ophthalmic disease or condition, for example affecting one or both eyes of the subject. The suspension formulations as described herein may be administered topically (e.g. to a tissue or organ surface) or may be administered by injection. The formulation may be administered parenterally, for example by injection, e.g. subcutaneous, or intramuscular injection. In one embodiment, the formulation may be administered by injection to the eye (ocular injection), or tissue of the eye. Methods of ocular injection applicable in the context of the disclosure may include intravitreal, suprachoroidal, juxtascleral, sub-conjunctival, intra-cameral, sub-retinal, sub-tenon, or periocular injection.

The use of the suspension formulations described in any one of the embodiments herein is also provided in the context of the present disclosure, for the manufacture or preparation of a medicament or medicine for said uses. Similarly, therapeutic uses as described in any one or combination of the embodiments described herein above may be featured in a method of treating a subject in need thereof, said method comprising the administration of the suspension formulation to said subject.

In yet a further aspect, the present disclosure may relate to a kit comprising a suspension formulation as defined herein and a container adapted for holding said formulation, and optionally a dispensing means.

Examples of dispensing means may be a dispensing means adapted for administration of the suspension formulation topically, or adapted for administration by injection, e.g. to the skin, eye, ear, nose, lung of a subject. Examples of dispensing means include, for example, a needle that is suitable or adapted for injection of the formulation, or an eye dropper which is adapted for dispensing the suspension formulation to the eye. In one embodiment, the container adapted for holding the suspension formulation may also be adapted for resuspension of the protein particles. The container may be suited or adapted to be mechanical agitated, such as by rotation or shaking, e.g. by hand (manually) or to a shaking means such as a shaker or rotator. The container according to any one of the embodiments herein may also be adapted for shaking at a frequency of up to 15 Hz. The container as described herein may also be adapted, or filled so as to provide sufficient headspace allowing for resuspension of the formulation. The container also may be adapted, in some embodiments, for administration of the formulation by injection, or by topical administration. In one embodiment, the container and optionally dispensing means are adapted for topical administration, or parenteral injection.

In one embodiment, the kit may be a pre-filled syringe comprising a syringe, which may function as a container adapted for holding the formulation and optionally a needle for injection. Said In yet a further embodiment, the syringe and dispensing means may be adapted for ocular injection, preferably for intravitreal, suprachoroidal, juxtascleral, sub-conjunctival, intra-cameral, sub-retinal, sub-tenon, or periocular injection. The kit may also further comprise instructions for use of the container or dispensing means and for administration of the suspension formulation and may be provided in a tangible or readable form such as an instruction leaflet or package label or insert.

The following list of numbered items are embodiments comprised by the present invention:
1. A suspension formulation comprising a protein particle suspended in a non-aqueous vehicle, wherein the particle comprises a protein and a stabilizing agent, and wherein the residual water content of the suspended protein particle is less than 1.0 wt % based on total weight of the particle.
2. The suspension formulation according to item 1, comprising a spray-dried or a lyophilized protein particle.
3. The suspension formulation according to item 2, comprising a spray-dried and vacuum-dried protein particle.
4. The suspension formulation according to any one of the preceding items, wherein the non-aqueous vehicle is a liquid at room temperature and/or is non-miscible with water.
5. The suspension formulation according to any one of the preceding items, wherein the non-aqueous vehicle comprises a semifluorinated alkane, a medium chain triglyceride (MCT), ethyl lactate, ethyl oleate, or mixtures thereof.
6. The suspension formulation according to any one of the preceding items, wherein the non-aqueous vehicle comprises one or more semifluorinated alkanes.
7. The suspension formulation according to any one of the preceding items, wherein the non-aqueous vehicle consists of one or more semifluorinated alkane, and optionally one or more pharmaceutically acceptable excipients.
8. The suspension formulation according to any one of items 6 or 7, wherein the one or more semifluorinated alkanes is a semifluorinated alkane of formula F(CF₂)ₙ(CH₂)ₘ, wherein n is an integer selected from 4 to 6 and m is an integer selected from 4 to 8.
9. The suspension formulation according to item 8, wherein the non-aqueous vehicle comprises, or consists of one or more semifluorinated alkanes selected from the group consisting of F4H4, F4H5, F4H6, F4H8, F6H4, F6H6, F6H8.
10. The suspension formulation according to any one of items 1 to 9, wherein non-aqueous vehicle is a vehicle selected from F4H5, F6H8, ethyl oleate and a medium chain triglyceride, or is a vehicle selected from F4H5 and F6H8.
11. The suspension formulation according to any one of the preceding items, wherein the residual water content of the suspended protein particle is less than 0.5 wt % based than the total weight of the particle.
12. The suspension formulation according to any one of the preceding items, wherein the residual water content of the suspended protein particle is in the range of 0.05 to 1.0 wt % based on total weight of the particle.
13. The suspension formulation according to any one of the preceding items, wherein the residual water content of the suspended protein particle is between 0.05 to 0.5 wt % based on the total weight of the particle.
14. The suspension formulation according to any one of the preceding items, wherein the total residual water content of suspension formulation is less than 1.0 mg/ml, or less than 0.5 mg/ml based on the total volume of the formulation.
15. The suspension formulation according to any one of the preceding items, wherein the relative weight ratio of the protein to stabilizing agent is in the range of 1:1 to 7:3.
16. The suspension formulation according to any one of the preceding items, wherein the stabilizing agent is selected from saccharides, polyols, amino acids, amines, surfactants, antioxidants, polymers, salts or combinations thereof
17. The suspension formulation according to item 16, wherein the stabilizing agent is selected from a saccharide, a polyol, an amino acid, an amine, a glycol, and an inorganic salt.
18. The suspension formulation according to any one of the preceding items, wherein the stabilizing agent is a saccharide, a polyol, a polysorbate or a combination thereof
19. The suspension formulation according to any one of the preceding items, wherein the stabilizing agent is a saccharide, preferably a saccharide selected from trehalose and sucrose.
20. The suspension formulation according to any one of the preceding items, wherein the protein has a molecular mass of between 10 to 300 kDa.
21. The suspension formulation according to any one of the preceding items, wherein the protein is selected from an antigen-binding polypeptide or protein, a vaccine and an enzyme.
22. The suspension formulation according to any one of the preceding items, wherein the protein is selected from antibody, preferably a monoclonal antibody or immunoglobulin (e.g. IgG), an antibody fragment, a fusion protein comprising an antibody fragment, an antibody-drug conjugate, and an enzyme.
23. The suspension formulation according to any one of the preceding items, wherein the protein is a chimeric, humanized or human monoclonal antibody.
24. The suspension formulation according to any one of the preceding items, wherein the protein is selected from the group consisting of a lysozyme and an antibody, (e.g. aflibercept, ziv-aflibercept, or bevacizumab).
25. The suspension formulation according to any one of the preceding items, wherein the protein is selected from the group consisting of aflibercept, ziv-aflibercept, and bevacizumab.
26. The suspension formulation according to any one of the preceding items, wherein the protein concentration is between 5 and 350 mg/ml.
27. The suspension formulation according to any one of the preceding items, wherein total solid content (TSC) of the formulation is between 10-500 mg/ml.
28. The suspension formulation according to any one of the preceding items, wherein the percentage of protein in respect of the total solid content (TSC) of the formulation is between 50-70%.
29. The suspension formulation according to any one of the preceding items, wherein the protein particles have a mean diameter of less than 30 μm, as determined by laser diffraction.
30. The suspension formulation according to any one of the preceding items, wherein at least 90% of the protein particles has a mean diameter of between 1 and 30 μm as determined by laser diffraction.
31. The suspension formulation according to any one of the preceding items, wherein the injection glide force is less than 35 N, preferably less than 25 N after storage at 40° C. for up to 12 months.
32. The suspension formulation according to item 31, wherein the non-aqueous vehicle comprises, or consists of semifluorinated alkane, preferably F4H5, or F6H8, and wherein the injection glide force is less than 15 N, preferably less than 15 N after storage at 40° C. for up to 12 months.
33. The suspension formulation according to any one of items 31 or 32, wherein the injection glide force is for a flow rate of 0.1 ml/s, and injection with a 1-mL syringe and 27G needle.
34. The suspension formulation according to any one of the preceding items, wherein the viscosity of the formulation, as measured by rotational viscometry at 25° C. is between 5 and 40 mPa·s.
35. The suspension formulation according to any one of the preceding items, wherein the protein particle consists of a protein, stabilizing agent, and optionally one or more excipients.
36. The suspension formulation according to any one of the preceding items, wherein the suspension formulation consists of a protein particle suspended in a non-aqueous vehicle, and wherein the protein particle consists of a protein and stabilizing agent, and optionally one or more excipients.
37. The suspension formulation according to any one of the preceding items, wherein the formulation further comprises one or more excipients, for example a surfactant (e.g. polysorbate 20, or polysorbate 80).
38. The suspension formulation according to any one of the preceding items, wherein the suspension formulation does not comprise, or is free of a surfactant and/or a preservative.
39. The suspension formulation according to any one of the preceding items, wherein:
   the non-aqueous vehicle comprises, or consists of a semifluorinated alkane, preferably selected from F4H5 or F6H8,
   the relative weight ratio of the protein to stabilizer agent in the protein particle is between 1:1 to 7:3,
   the residual water content of the protein particle is less than 0.5 wt %; preferably less than 0.3 wt % relative to the total weight of the protein particle; and wherein the total solid content of the formulation is no more than about 300 mg/ml.
40. The suspension formulation according to item 39, wherein the total solid content of the formulation is 300 mg/ml.
41. The suspension formulation according to item 39, wherein the total solid content of the formulation is no more than about 100 mg/ml.
42. The suspension formulation according to any one of items 39 to 41, wherein residual water content of the formulation is less than 0.4 wt %, preferably less than 0.25 wt % based on the total weight of the formulation.
43. The suspension formulation according to any one of items 39 to 42, wherein the protein particle is a spray-dried protein particle, preferably a spray-dried and a vacuum dried protein particle.
44. The suspension formulation according to any one of items 39 to 43, wherein:
   the non-aqueous vehicle consists of a semifluorinated alkane selected from F4H5 or F6H8, and optionally one or more excipients;
   the protein particle is a spray-dried particle comprising a protein, a stabilizer agent, and optionally one or more further excipients; wherein the protein is a monoclonal antibody, or is selected from the group consisting of a lysozyme, an immunoglobulin, aflibercept, ziv-aflibercept, or bevacizumab; and wherein the stabilizer agent is a saccharide, preferably selected from sucrose and trehalose;
   the relative weight ratio of the protein to stabilizer agent in the protein particle is between 1:1 to 7:3,
   the residual water content of the protein particle is less than 0.5 wt %, preferably less than 0.3 wt % relative to the total weight of the protein particle and wherein
   the total solid content of the formulation is no more than about 300 mg/ml.
45. A suspension formulation as according to any one of items 1-44 for use as a medicine.
46. The suspension formulation for use according to item 45 wherein the use comprises treatment of a disease or condition affecting the skin, eye, ear, nose, or lung in a subject in need thereof
47. The suspension formulation for use according to item 46, wherein the use comprises the treatment of an ophthalmic disease or condition.
48. The suspension formulation for use according to any one of items 45 to 47, wherein the formulation is administered topically or by injection.
49. A suspension formulation for use according to any one of items 45 to 48, wherein the formulation is administered by injection (e.g. subcutaneous, or intramuscular injection); or is administered by injection to the eye (ocular injection), preferably intravitreal, suprachoroidal, juxtascleral, sub-conjunctival, intra-cameral, sub-retinal, sub-tenon, or periocular injection.
50. Use of a suspension formulation according to any one of items 1 to 44 in the manufacture of a medicament for the treatment of a disease or condition in a subject or patient.
51. The use according item 50, wherein the medicament is for use in the treatment of a disease or condition affecting the skin, eye, ear, nose, or lung in a subject.
52. The use according to item 51, wherein the medicament is for use in the treatment of an ophthalmic disease or condition.
53. The use according to any one of items 50 to 52, wherein the medicament is a topically administered medicament, or is a medicament formulated or adapted for injection.
54. The use according to any one of items 50 to 53, wherein the medicament is administered by injection (e.g. subcutaneous, or intramuscular injection); or is administered by injection to the eye (ocular injection), preferably intravitreal, suprachoroidal, juxtascleral, sub-conjunctival, intra-cameral, sub-retinal, sub-tenon, or periocular injection.
55. A method of treating a disease or condition, the method comprising administering a suspension formulation according to any one of items 1 to 44 to a subject in need thereof
56. The method according to item 55, wherein the disease or condition is a disease or condition affecting the skin, eye, ear, nose, or lung of the subject.
57. The method according to item 55 or 56, wherein the disease or condition is an ophthalmic disease or condition.
58. The method according to any one of items SS to 57, wherein the suspension formulation is administered topically, or is administered by injection.
59. The method according to any one of items SS to 58, wherein the suspension formulation is administered by injection (e.g. subcutaneous, or intramuscular injection); or is administered by injection to the eye (ocular injection), preferably intravitreal, suprachoroidal, juxtascleral, sub-conjunctival, intra-cameral, sub-retinal, sub-tenon, or periocular injection.
60. A suspension formulation as defined in any one of items 1-44 obtained or obtainable by a process comprising the steps of:
   a) spray-drying or lyophilizing an aqueous solution comprising the protein and the stabilizing agent to obtain protein particles,
   b) drying the protein particles obtained in step a) to obtain residual water content of less than 1.0 wt %, or less than 0.5 wt %, based on the weight of the particle, and
   c) suspending the protein particles of step b) in the non-aqueous vehicle
   d) and optionally, homogenizing the suspension formulation, preferably by high-shear homogenization, milling, or ultrasonication.
61. A suspension formulation as defined in any one of items 1-44 obtained or obtainable by a process comprising the steps of:
   a) spray-drying or lyophilizing an aqueous solution comprising the protein and the stabilizing agent to obtain protein particles,
   b) vacuum drying the protein particles obtained in step a), and
   c) suspending the protein particles of step b) in the non-aqueous vehicle;
   d) and optionally, homogenizing the suspension formulation, preferably by high-shear homogenization, milling, or ultrasonication.
62. A suspension formulation obtainable according to the process of item 60 or 61 comprising in step a), spray drying an aqueous solution comprising the protein and stabilizing agent to obtain protein particles.
63. The suspension formulation obtainable according to any one of items 60 to 62, wherein the relative weight ratio of the protein to the stabilizing agent is between 1:1 to 7:3.
64. The suspension formulation obtainable according to the process of any one of items 60 to 63, wherein the spray drying in step a) is conducted using cyclone spray dryer.
65. The suspension formulation obtainable according to any one of items 60 to 64, wherein step b) drying is vacuum drying, preferably wherein the vacuum drying is conducted at a temperature of between 15-40° C., at a pressure of between 0.01-100 mbar.
66. The suspension formulation obtainable according to any one of items 60 to 65, wherein step b) is conducted for at least 12 hours, or at least 24 hours.
67. The suspension formulation obtainable according to any one of items 60 to 66, wherein step b) vacuum drying is conducted to obtain particles with a residual water content of less than 1.0 wt %, or less than 0.5 wt %, based on the weight of the particle.
68. A process for the manufacture of a suspension formulation as defined in any one of items 1-44, the method comprising the steps of:
   a) spray-drying or lyophilizing an aqueous solution comprising the protein and the stabilizing agent to obtain protein particles,
   b) drying the protein particles obtained in step a) to obtain particles comprising a residual water content of less than 1.0 wt %, or less than 0.5 wt %, based on the weight of the particle, and
   c) suspending protein particles of step b) in the non-aqueous vehicle, and optionally;
   d) homogenizing the suspension formulation, preferably by high-shear homogenization, milling, or ultrasonication.
69. A process for the manufacture of a suspension formulation as defined in any one of items 1-44, the method comprising the steps of:
   a) spray-drying or lyophilizing an aqueous solution comprising the protein and the stabilizing agent to obtain protein particles,
   b) vacuum drying the protein particles obtained in step a), and
   c) suspending protein particles of step b) in the non-aqueous vehicle, and optionally;
   d) homogenizing the suspension formulation, preferably by high-shear homogenization, milling, or ultrasonication.
70. The process according to any one of items 68 or 69, comprising spray-drying in (step a) an aqueous solution comprising the protein and stabilizing agent to obtain protein particles.
71. The process according to any one of items 68 to 70 wherein the relative weight ratio of the protein to the stabilizing agent is between 1:1 to 7:3.
72. The process according to any one of items 68 to 71, wherein step b) drying is vacuum drying, preferably vacuum drying conducted at a temperature of between 15-40° C., at a pressure of between 0.01-100 mbar.
73. The process according to any one of items 68 to 72, wherein step b) is conducted for at least 12 hours, or at least 24 hours.
74. The process according to any one of items 68 to 73, wherein the step b) vacuum drying is conducted to obtain particles with a residual water content of less than 1.0 wt %, or less than 0.5 wt %, based on the weight of the particle.
75. A kit comprising a suspension formulation as defined in any one of items 1-44, and a container adapted for holding said formulation, and optionally a dispensing means.
76. The kit according to item 75, wherein the container adapted for holding the formulation is a pre-filled syringe, or wherein the kit further comprises a syringe, and optionally a dispensing means, preferably a needle adapted for injection of the formulation.
77. The kit according to item 76, wherein the syringe and dispensing means is adapted for ocular injection, preferably for intravitreal, suprachoroidal, juxtascleral, sub-conjunctival, intra-cameral, sub-retinal, sub-tenon, or periocular injection.
78. An administration device comprising a suspension formulation as defined in any one of items 1 to 44.
79. An administration device according to item 78, wherein the administration device is adapted for topical administration, or administration of the suspension formulation by injection.
80. An administrative device according to item 78 or 79, wherein the administrative device comprises a syringe, and optionally a needle.
81. An administration device of item 78 to 80, wherein the administration device is adapted for subcutaneous administration of a suspension formulation as defined in any one of items 1 to 44.
82. The process according to items 68 to 74, further comprising a step of selecting the protein particles with a predetermined particle size to be suspended in the non-aqueous vehicle.
83. The process according to item 82, wherein the predetermined particle size is characterized by a distribution of at least 90% of the particles having a mean diameter of between 1 and 15 μm, between 1 and 30 μm, or between 1 and 50 μm, or is characterized by a mean diameter of less than 50 μm, less than 30 μm, less than 15 μm, between 1 and 15 μm, between 1 and 30 μm or between 1 and 50 μm, each as determined by laser diffraction.

The following examples serve to illustrate the invention, however should not to be understood as restricting the scope of the invention.

### EXAMPLES

### Example 1—Preparation of Suspension Formulations

Materials—Lysozyme bulk solutions were prepared by dissolution of pure lysozyme (lys) (Ovobest, Neuenkirchen-Voerden, Germany) in 10 mM histidine buffer at pH 6.0. A model monoclonal antibody of the IgG1 type (mAb) in 25 mM histidine 1.6 mM glycine buffer pH 6.0 at 56 mg/ml was used. The mAb was produced in CHO cells and has an ε280 nm of 1.49 ml g⁻¹ cm⁻¹. Samples of Bevacizumab (Beva) (marketed product Avastin) was acquired from a local pharmacy. Formulations were prepared in highly purified water prepared with an ELGA Purelab system (ELGA LabWater, Celle, Germany) using Trehalose (Tre) (Hayashibara Co. Ltd, Okayama, Japan), Sucrose (Suc), L-Histidine, L-Histidine-monohydrochloride monohydrate (Sigma-Aldrich, St. Louis, USA) and Polysorbate 20 (PS20) (Merck KGaA, Darmstadt, Germany). Perfluorobutylpentane (F4H5) and perfluorohexyloctane (F6H8) were provided by Novaliq GmbH (Heidelberg, Germany). Further, medium chain triglycerides (MCT) (Miglyol 812 by Caesar & Loretz GmbH, Hilden, Germany) and ethyl oleate (EO) (Sigma-Aldrich, St. Louis, USA) were also tested as suspension vehicles.

### Analytical Methods—

UV-Vis—Protein concentrations were measured with a NanoDrop 2000 spectrophotometer (Thermo Scientific, Waltham, USA) at 280 nm.

Scanning electron microscopy (SEM)—Powders were investigated on self-adhesive carbon tapes positioned on aluminium stubs using a FEI Helios G3 UC (Thermo Fisher Scientific, Waltham, USA). Suspension-milled powders (in F6H8) were pipetted directly on the carbon tape and dried in a VTS-2 vacuum drier (Memmert, Schwabach, Germany) for 24 h at 10 mbar.

Laser diffraction—Particle size distribution was analysed in isooctane containing 1% Span 80, as a dispersing medium, using the Laser Diffraction Particle Size Analyzer LA-960 by Horiba (Horyiba, Kyoto, Japan). In order to investigate initial dispersing quality, no additional dispersion step in the dispersing medium was conducted. Particle sizes after storage were analysed after an additional dispersing step using the ultrasonic homogenizer Bandelin Sonoplus (BANDELIN electronic GmbH & Co. KG, Berlin, Germany) with a MS 72 probe (30 s 20% intensity). The additional step was conducted, in order to distinguish between agglomerated particles and particles which were already sintered together.

Light microscopy—Light microscopy was performed using a Keyence Digital microscope VHX 500F (Keyence Corporation, Osaka, Japan) with a VH-Z100R lens at 200× magnification. For analysis, suspension formulations were dispersed in MCT to a concentration of 5 mg/ml. The resulting suspension formulation was then transferred onto a glass slide and was subsequently investigated.

### Suspension Formulation Preparation

Spray-drying—Feed solutions for spray-drying with a total solid content of 7.5% (m/V) containing trehalose or sucrose at different protein to stabilizer ratios and optionally a surfactant (e.g. polysorbate 20) were prepared. Protein particles based on lysozyme (lys), a model monoclonal antibody (mAb) and bevacizumab (beva) were prepared. Protein to stabilizer ratios described are based on mass ratios. All solutions were prepared in a 10 mM histidine buffer at pH 6.0.

Spray-drying was conducted using a Büchi B290 (Büchi AG, Flawil, Switzerland) equipped with a high-efficient cyclone according to the manufacturer's recommendations (e.g. nozzle diameter 0.7 mm, drying air flow rate 35 m3/h, atomizing air flow rate 414 L/h) keeping the outlet temperature at 70° C.

The protein-containing particles obtained from spray-drying were transferred in 10R type 1 glass vials (MGlas AG, Muennerstadt, Germany) and a lyophilization stopper was attached (Helvoet Pharma, Tilburg, Netherlands). An additional drying step, i.e. vacuum drying, was conducted using a Christ 2-6D (Martin Christ Gefriertrocknungsanlagen GmbH, Osterode, Germany) at 32° C., 0.1 mBar for 24 h.

Handling of the protein-containing particles was conducted under nitrogen environment to prevent water uptake of the hygroscopic powders.

Suspension formulations were prepared from the spray-dried and vacuum dried protein-containing formulations. The suspension formulations were prepared in either 2R (Schott AG, Mainz, Germany) (Beva), 6R (mAb), or 20R (Lys) (MGlas AG, Muennerstadt, Germany) type 1 glass vials glass vials at different concentrations by addition of the respective vehicle to the calculated amount of spray-dried protein-containing particles. Suspensions were then homogenized using either a high-shear homogenizer Ultraturrax T10 (IKA-Werke GmbH & Co. KG, Staufen, Germany) (sh; 2 min/20 000 rpm) or in an VWR Ultrasonic cleaner (VWR, Radnor, USA) cooled with ice (us; 20 min with additional shaking by hand after 5, 10 and 15 min).

Concentrations of the suspension formulations are based on the total solid content (TSC) of the suspension formulations.

The amount of residual water of the protein-containing particles or the suspension formulations was analysed using the Karl-Fischer-Titrator Aqua 40.00 (Analytik Jena AG; Jena, Germany) equipped with a head space module at a chamber temperature of 100° C.

Exemplary suspension formulations 1a-15b described according to these general methods are described in Table 1. In Table 1, the protein to stabilizer ratios of each formulation, as well as the determined residual water content of the particles as well as the water content of the suspension formulations are described.

TABLE 1

| | | | | | | | | Residual | Residual |
|---|---|---|---|---|---|---|---|---|---|
| | | | | | | | | water | water |
| | | | | | | | | content, | content, |
| | | | | Protein | | Total | | protein- | sus- |
| | | | | to | | solids | | containing | pension |
| For- | | | Sus- | stabilizer | Homo- | content | Additional | particle | for- |
| mulation, | | | pension | ratio | genization | (TSC) | drying | [%] | mulation |
| No. | Protein | Stabilizer | vehicle | (m:m) | technique ³ | [mg/ml] | step | [mg/ml] | [mg/ml] |
| 1a | lysozyme | trehalose | F4H5 | 70:30 | us | 100 | yes | 0.1 | 0.1 |
| 1b | lysozyme | trehalose | F6H8 | 70:30 | us | 100 | yes | 0.1 | 0.1 |
| 1c | lysozyme | trehalose | EO | 70:30 | us | 100 | yes | 0.1 | 0.1 |
| 1d | lysozyme | trehalose | MCT | 70:30 | us | 100 | yes | 0.1 | 0.1 |
| 2 | lysozyme | trehalose | F6H8 | 70:30 | us | 300 | yes | 0.1 | 0.3 |
| 3 | lysozyme | trehalose¹ | F6H8 | 70:30 | us | 100 | yes | 0.2 | 0.2 |
| 4 | lysozyme | trehalose | F6H8 | 50:50 | us | 100 | yes | 0.2 | 0.2 |
| 5a | lysozyme | sucrose | F6H8 | 50:50 | us | 100 | no | 3.6 | 3.6 |
| 5b | lysozyme | sucrose | F6H8 | 50:50 | us | 100 | yes | 0.2 | 0.2 |
| 6a | lysozyme | sucrose | EO | 50:50 | us | 100 | no | 3.6 | 3.6 |
| 6b | lysozyme | sucrose | EO | 50:50 | us | 100 | yes | 0.2 | 0.2 |
| 7a | mAh | sucrose | F4H5 | 50:50 | us | 100 | no | 4.2 | 4.2 |
| 7b | mAb | sucrose | F4H5 | 50:50 | us | 100 | yes | 0.1 | 0.1 |
| 8a | mAb | sucrose | F6H8 | 50:50 | us | 100 | no | 4.2 | 4.2 |
| 8b | mAb | sucrose | F6H8 | 50:50 | us | 100 | yes | 0.1 | 0.1 |
| 9a | mAb | sucrose | EO | 50:50 | us | 100 | no | 4.2 | 4.2 |
| 9b | mAb | sucrose | EO | 50:50 | us | 100 | yes | 0.1 | 0.1 |
| 10a | mAb | sucrose | MCT | 50:50 | us | 100 | no | 4.2 | 4.2 |
| 10b | mAb | sucrose | MCT | 50:50 | us | 100 | yes | 0.1 | 0.1 |
| 11a | mAb | sucrose | F6H8 | 50:50 | sh | 100 | no | 4.2 | 4.2 |
| 11b | mAb | sucrose | F6H8 | 50:50 | sh | 100 | yes | 0.1 | 0.1 |
| 12a | mAb | sucrose | MCT | 50:50 | sh | 100 | no | 4.2 | 4.2 |
| 12b | mAb | sucrose | MCT | 50:50 | sh | 100 | yes | 0.1 | 0.1 |
| 13a | mAb | trehalose | F6H6 | 50:50 | us | 100 | no | 3.9 | 3.9 |
| 13b | mAb | trehalose | F6H6 | 50:50 | us | 100 | yes | 0.1 | 0.1 |
| 14a | Bevacizumab | sucrose | F6H8 | 50:50 | us | 100 | no | 3.5 | 3.5 |
| 14b | Bevacizumab | sucrose | F6H8 | 50:50 | us | 100 | yes | 0.1 | 0.1 |
| 15a | Bevacizumab | sucrose | EO | 50:50 | us | 100 | no | 3.5 | 3.5 |
| 15b | Bevacizumab | sucrose | EO | 50:50 | us | 100 | yes | 0.1 | 0.1 |
| 16 | Bevacizumab | trehalose² | F6H8 | 70:30 | us | 8 | yes | 0.1 | 0.008 |

¹Contains 0.1% polysorbate20;
²contains 0.5% polysorbate20;
³ us = ultrasonic homogenization,
sh = high-shear homogenization

Particle Redispersion after Suspension Preparation

Obtaining initial homogeneous suspension formulations is of high importance for the preparation of injectable suspension formulations. Suspension formulations may be homogenized using a suitable dispersing technique, such as a high-shear homogenizer, suspension milling or an ultrasonification technique. The suspension formulations herein were prepared using an ultrasound bath cooled with ice or alternatively using a high-shear homogenizer.

It was observed that the quality of the suspensions in terms of dispersibility was higher for suspension formulations prepared with additional drying step after spray-drying.

Suspension formulations prepared with the additionally dried protein-containing particles were prepared using the ultrasound bath homogenization method were found to be readily dispersible in F4H5, F6H8, EO and MCT (Formulation No 1a-d). This was similarly observed for higher concentration formulations dispersed in F6H8 as the liquid vehicle (2; 300 mg/ml), PS20 (polysorbate 20) containing formulations (3), as well as formulations with a higher trehalose content (4).

It was observed, directly after their preparation, that suspensions prepared with protein particles having undergone an additional drying step (for example by vacuum drying) had an impact on initial dispersion quality in terms of a more homogeneous particle size distribution as well as overall smaller particle sizes.

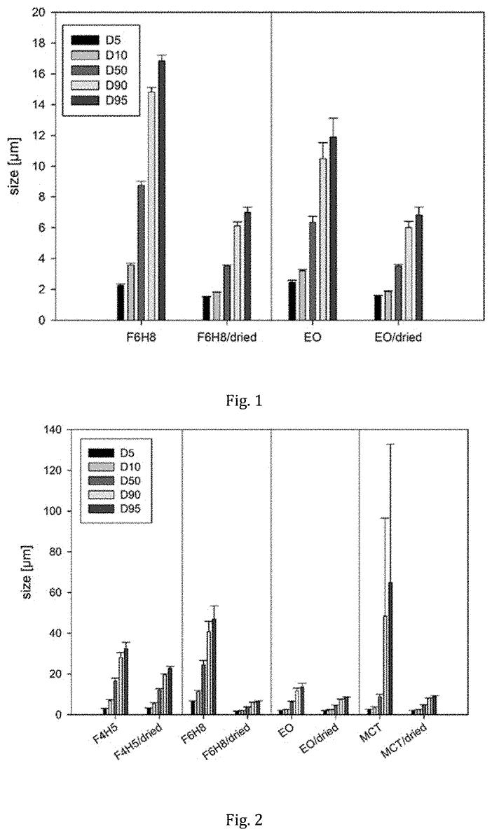

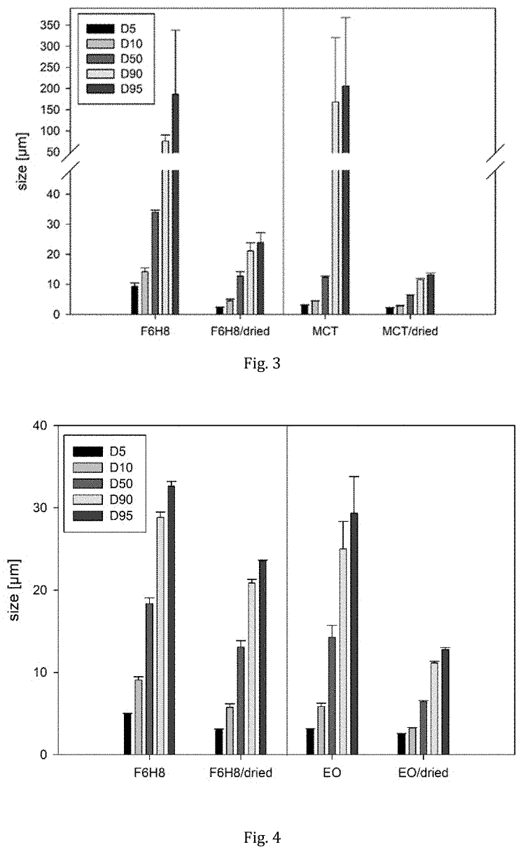

As depicted in FIG. 1 (formulations 5a, 5b, 6a, and 6b), FIG. 2 (formulations 7a, 7b, 8a, 8b, 9a, 9b, 10a, and 10b) and FIG. 3 (formulations 11a, 11b, 12a, 12b), and FIG. 4 (14a, 14b, 15a, 15b) suspension formulations prepared from exemplary protein particles which were prepared without an additional vacuum drying step and which had a residual moisture content were, in contrast to the suspension formulations prepared using protein particles which were vacuum dried and which had a low residual water content (e.g. of less than 0.5 wt %) see Table 1, were found to have a poorer initial dispersion quality (e.g. less homogenous particle size distribution, overall larger median (d50) particle diameter values), regardless of whether ultrasound bath or the high-shear homogenizer was used for homogenization of the suspensions. The laser diffraction results were also further confirmed by light microscopy.

### Example 2—Stability Studies

Suspension formulations prepared according to Example 1 were filled in N 13-2 glass vials (Beva 9a-10b; 0.4 ml) (Macherey-Nagel, Duren, Germany), 2R glass vials (Lys 1a-5d,11a-11d; mAb:Suc 6a-7d; 1 ml) or in Terumo Plajex (mAb:Tre 8a-8d; 1 ml) (Terumo, Tokyo, Japan) prefillable syringes. Filling was conducted by hand using a B. Braun Injekt (B. Braun AG, Melsungen, Germany) syringe with a Terumo Agani 30G (Terumo, Tokyo, Japan) needle attached (inner diameter ≈160 μm) in order to ensure initial injectability. Vials were closed using 13 or 20 mm Teflon coated injection stoppers and were then manually capped using 10R caps (Westpharma, Exton, USA).

### Particle Size Stability

An increase in particle size of a suspension formulation over time may, for example for formulations which are to be administered to a subject by injection, lead to increased likelihood of needle clogging, and may also result in altered release kinetics.

Particle size stability was studied in suspension formulations comprising lysozyme and a model mAb and with either sucrose or trehalose as a stabilizer, and prepared according to the general method described in Example 1 and stored at 5-8° C., 25° C. and 40° C.

Model mAb/Sucrose Particles Suspended in F4H5 and F6H8

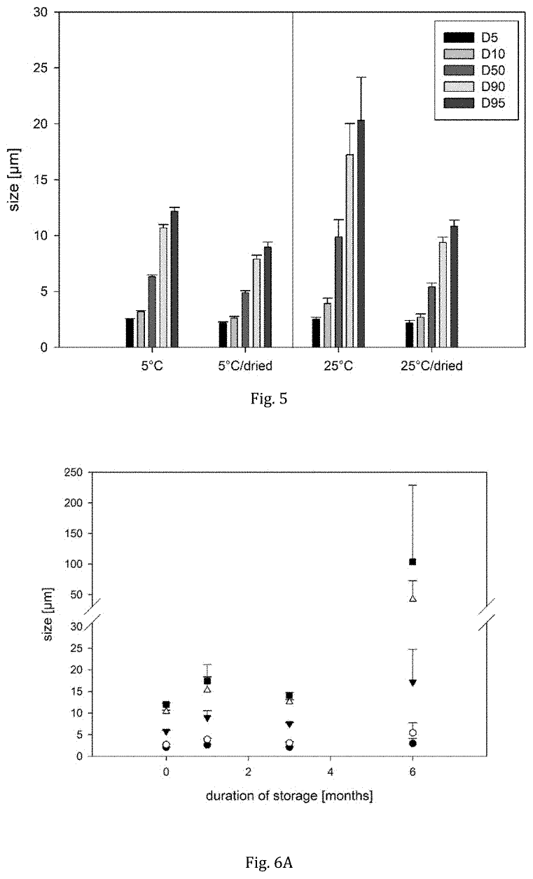

It was observed that the storage and aging of suspension formulations comprising protein particles of model mAb and sucrose suspended in F6H8 under cold conditions (5-8° C.) and at room temperature conditions of 25° C. for 6 months resulted in no significant changes in respect of particle size distribution for all tested formulations. FIG. 5 depicts particle size distribution of suspension formulations, from left to right, of suspension formulation 8a prepared from protein particles which were not subjected to vacuum drying (mAb:Suc 50:50; TSC=100 mg/ml), after storage for 6 months at 5° C., suspension formulation 8b (mAb:Suc 50:50; TSC=100 mg/ml) prepared from vacuum dried protein particles after 6 months storage at 5° C., of suspension formulation 8a after 6 months storage at 25° C., and suspension formulation 8b after 6 months storage at 25° C.

Unexpectedly, however, it was observed that under higher temperature stress conditions of 40° C., that suspension formulations containing the model mAb protein particles, which were prepared by spray drying and additionally vacuum-drying, and having less than 1.0 wt % residual water content did not undergo any significant increases or changes in particle size when stored for up to 6 months. In contrast, it was observed that the suspension formulations containing mAb particles which were not subjected to the vacuum drying step during particle preparation had drastic increases in particle size at the 6-months of storage at 40° C.

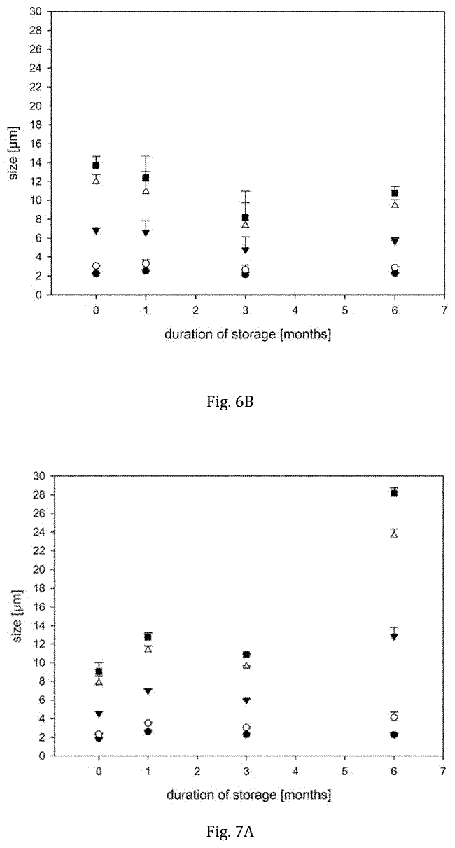

FIGS. 6A and 6B depict particle size distributions of suspension formulations of protein particles comprising a model mAb and sucrose in F4H5 as the liquid vehicle after storage at 0, 1, 3 and 6 months storage at 40° C.

FIG. 6A depicts the particle size distributions of suspension formulation 7a (mAb:Suc 50:50; TSC=100 mg/ml) which are prepared from particles which have not been subjected to vacuum drying, and which contained about 4.2 wt % residual water content. FIG. 6B depicts the particle size distributions of suspension formulation 7b (mAb:Suc 50:50; TSC=100 mg/ml) which comprise about 0.1 wt % residual water content.

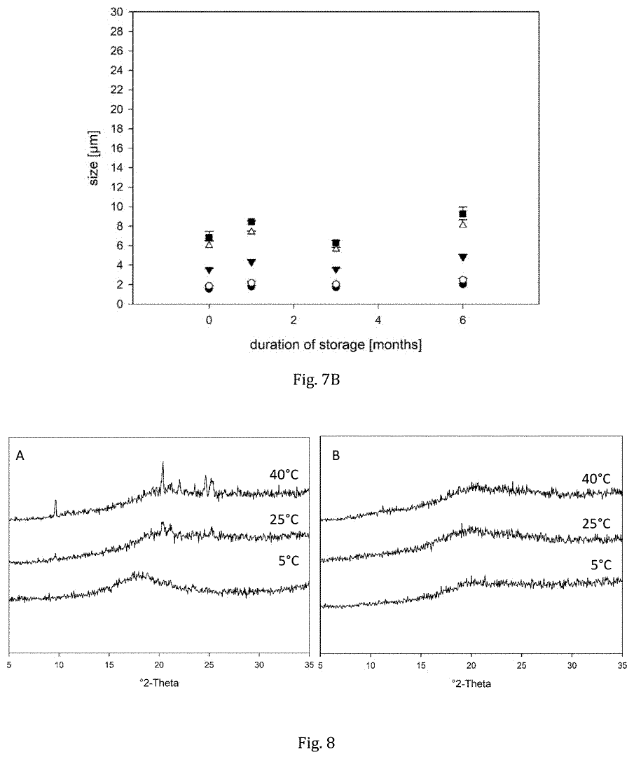

FIGS. 7A and 7B depict particle size distributions of suspension formulations of suspension formulations of protein particles comprising a model mAb and sucrose in F6H8 as the liquid vehicle after 0, 1, 3 and 6 months storage at 40° C.

FIG. 7A depicts the particle size distributions of suspension formulation 8a (mAb:Suc 50:50; TSC=100 mg/ml), which was prepared from particles which were not subjected to vacuum drying, and which contained about 4.2 wt % residual water content. FIG. 7B depicts the particle size distribution of suspension formulation 8b (mAb:Suc 50:50; TSC=100 mg/mL) which comprises only about 0.1 wt % residual water content. Particle size distributions D5 (●), D10 (◯), D50 (♦), D90 (Δ) and D95 (▪) values.

Consistent particle size distribution was observed for both formulations comprising protein particles prepared using the spray-drying and vacuum drying process as described in Example 1 and having lower residual water content where either F4H5 or F6H8 was used as the liquid vehicle (FIGS. 6B, 7B) over a 6-month period storage at 40° C., whereas in comparison, the particle size distribution was significantly changed at 6-months for the formulations prepared using the protein particles and formulations with higher residual water content, when no additional drying step was implemented in the process (FIGS. 6A, 7A).

SEM analysis of the formulation samples revealed the formation of needle-like structures, indicating crystallization effects as a root cause for the increase in the measured particle size. This assumption was further confirmed by XRD analysis of the formulation 8a (F6H8, mAb:Suc 50:50; TSC=100 mg/ml) showing peaks of crystalline sucrose (FIG. 8, spectra A). Crystallinity in XRD spectra of 8a particles was also seen after storage for 6 months at 25° C., although not after storage for 6 months at 5° C. Suspension formulations prepared from dried protein-containing particles (formulation 8b) on the other hand were found to remain in the amorphous state even after storage at 40° C. for 6 months (FIG. 8, spectra B).

The recrystallization of the stabilizing agent such as sucrose may not only has a negative impact in respect of particle size stability, but also on protein stability as its crystallinity may affect its function to stabilize the protein. The formulation as well as storage of protein particles prepared as described above, and having a reduced water-content of less than 1.0 wt % relative to the weight of the protein particle, for example of about 0.1 wt % or less, and in a liquid non-aqueous vehicle such as F4H5 or F6H8 may thus be advantageous in case of potential adverse storage situations such as loss of cold-chain or temperature control.

No change in particle size was found for suspension formulations prepared from the spray and vacuum-dried mAb-containing particles, which were stored in a prefillable COP syringe.

### Lysozyme/Trehalose in F6H8

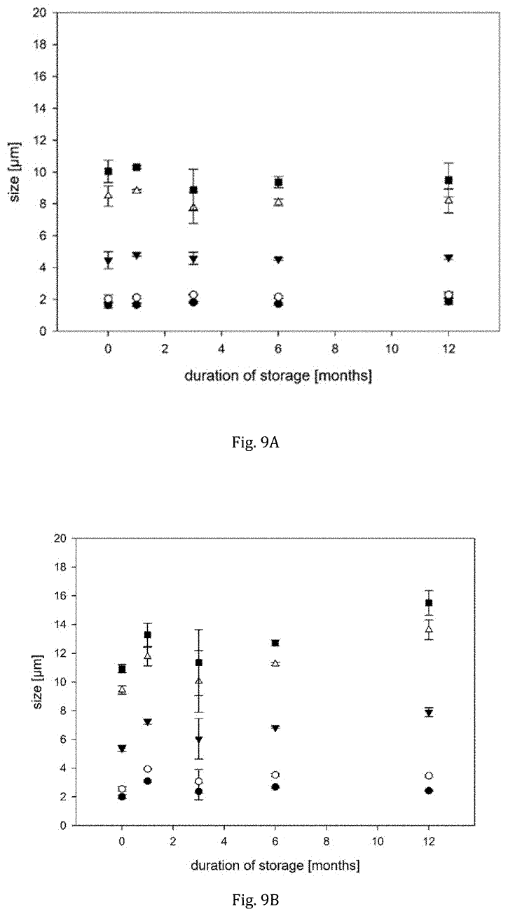

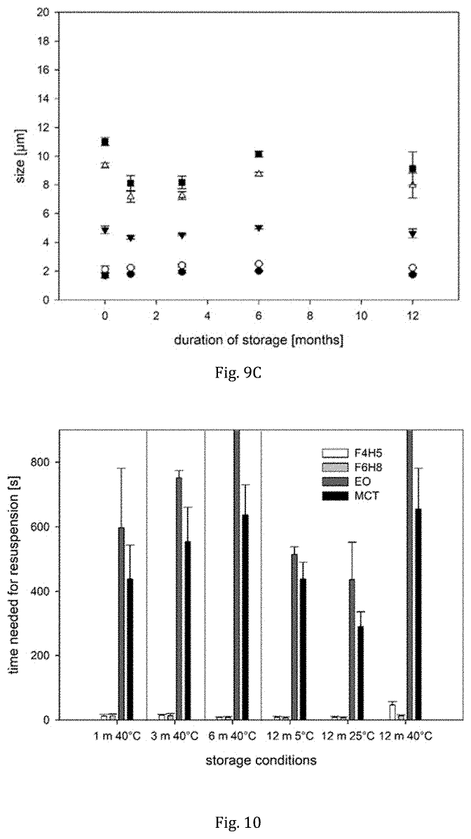

Particle size stability of lysozyme/trehalose particles suspended in F6H8 was also studied. FIGS. 9A, 9B, 9C depict the particle size stability of formulations prepared with spray-dried and vacuum dried particles comprising lysozyme and trehalose at different ratios, suspended in F6H8 as the liquid vehicle after 0, 1, 3, 6 and 12 months storage at 40° C. FIG. 9A depicts particle size distributions of suspension formulation 2 (Lys:Tre 70:30; TSC=300 mg/ml). FIG. 9B depicts particle size distributions of suspension formulation 3 (PS20 containing Lys:Tre 70:30 formulation; TSC=100 mg/ml). FIG. 9C depicts particle size distribution of suspension formulation 4 (Lys:Tre 50:50; TSC=100 mg/ml).

As shown in these Figures, it was also observed for protein particles comprising lysozyme and trehalose as stabilizer, and at differing concentrations that there was no difference in particle size even after 12 months of storage at 40° C. for these formulations. These results were further confirmed by light microscopy and SEM. Pictures taken with the SEM further showed that that there was no change of the particle morphology or sintering of single particles occurred during 12 months of storage at 40° C.

### Resuspendability

Resuspendability, which may also be referred to as redispersibility of some of the suspensions prepared according to Example 1 was tested using two different methods.

The resuspendability of a suspension formulation is another measure of its physical stability. The European Pharmacopoeia (Ph. Eur.) defines a general standard for suspensions, whereby suspensions need to be re-dispersible by gentle manual shaking. In particular, resuspension of a suspension formulation should not take too long, in order to provide for easy administration by the medical personnel or even by a patient themself. If a suspension formulation is intended to be provided as a kit or administration means such as a form of a prefillable syringe, the physical attribute of resuspendability is even more significant, due to the generally reduced head space volume available for resuspending the formulation. An unstable suspension formulation is one which cannot be fully resuspended or redispersed to its original characteristics. A suspension is not stable, for example, if the formation of particle aggregates, including visually observable aggregates such as floats, sediments or deposits in the container in which the formulation is stored and which may have formed over time on standing or storage, cannot be fully re-dispersed.

The first test (rotation method) for resuspendability was conducted using a SU1100 vertical rotator (Sunlab, Mannheim, Germany), at a rotation speed of 25 rpm. The time until visual resuspension was achieved was measured. The experiment was terminated after 15 min. The second method (shaking method) was performed using a Retsch swing mill MM 400 (Retsch GmbH, Haan, Germany). For this purpose, the vial containing the suspension formulation, was fixed and shaken at a constant frequency for 30 s. If no resuspension was visible, this step was repeated with a 2.5 Hz higher frequency (starting frequency: 5 Hz; maximum frequency: 30 Hz).

### Results

### Lysozyme/Trehalose Suspensions

Suspension formulations 1a, 1b, 1c, and 1d comprising protein particles comprising lysozyme and trehalose particles stored at 5° C., 25° C. and 40° C. were tested for resuspendability using a vertical shaker (vertical rotation at 25 rpm). As depicted in FIG. 10, the protein-containing suspension formulation comprising F4H5 or F6H8 as suspension vehicle were found to be readily resuspended after several seconds, even the samples stored at 40° C. for 12 months. Suspensions based on EO or MCT vehicles needed longer resuspension times of several minutes. Also in contrast to the semifluorinated alkanes, EO and MCT suspensions were observed to have a much lower sedimentation volume after storage and formed more dense cakes.

Resuspension tests conducted on formulations containing higher concentrations of protein particles (2); Lys:Tre 70:30; total solids content (TSC) 300 mg/ml), Polysorbate 20 (3; Lys:Tre 70:30; total solids content (TSC) 100 mg/ml; 0.1% polysorbate 20) or higher stabilizer concentrations (4; Lys:Tre 50:50; 100 mg/ml) in the vehicle F6H8 were also found to be readily redispersible, i.e. in less than a minute.

### Lysozyme/Sucrose Suspensions

Suspension formulations containing lysozyme and sucrose particles were also studied with respect to their resuspendability after storage over a period of 12 months at 5° C., 25° C. and 40° C., using either vertical rotation method or manual shaking method.

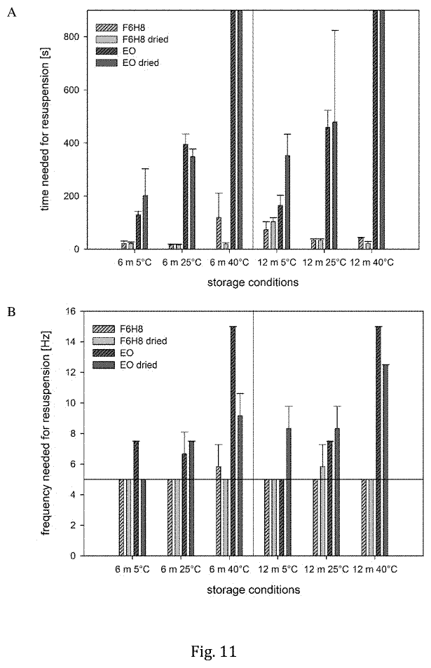

Similarly as observed for lysozyme/trehalose particles, it was observed that Lys:Suc 50:50 particles (TSC=100 gm/ml) in F6H8 (formulations 5a, 5b, 6a, and 6b) are generally more quickly resuspended using vertical rotation, compared to suspensions where the vehicle is EO (FIG. 11, graph A). Acceptable redispersion times were observed for EO-based formulations stored at lower temperatures, whereas for F6H8-based formulations, the resuspendability remained essentially consistently fast at all the various storage temperatures.

Using the shaking method for testing resdispersion, which simulates the shaking by hand the frequency needed for resuspension was also tested for these formulations (FIG. 11, graph B). The lysozyme suspensions in F6H8 were easy re-dispersible at a frequency of 5 Hz, which is the frequency an average person would use for this operation. EO formulations required higher frequencies of up to 15 Hz.

Model mAb/Sucrose Suspensions

Resuspension tests were also conducted for suspension formulations 7a, 7b, 8a, 8b, 9b, 10b (mAb:Suc 50:50; TSC=100 mg/ml) as described in Table 1, aged over a 6 month period at 5° C., 25° C., and 40° C.

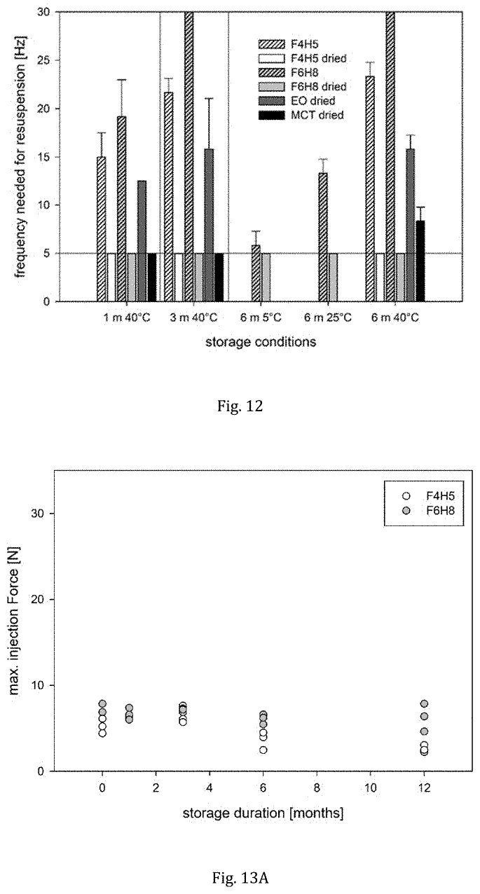

It was observed (FIG. 12) that the suspensions containing mAb-containing particles (mAb:sucrose 50:50), which were prepared without the additional step of drying (formulations 7a, 8a), were not as readily re-suspended, compared to for example the lysozyme/sucrose particles described above, and that formation of a particle scaffold was also observed. In contrast, however, the suspension formulations which were prepared with the mAb:sucrose particles with low residual water content (formulations 7b, 8b) were readily redispersible using the shaking method. Notably, these formulations were also re-dispersable after pro-longed storage at higher temperatures i.e. at 40° C.

### Syringeability and Injectability

Syringeability, or the overall ease in which formulations may be withdrawn and filled into the volume of a syringe, of the suspensions prepared according to Example 1 was tested. Syringeability was tested manually. A 23G needle (Terumo) was attached to a 1 ml B. Braun Inject F single-use syringe (B. Braun AG, Melsungen, Germany). The suspension formulations were then tested for syringeability by moving the plunger to the end of the syringe. The removable volume was measured.

Injectability is also important parameter for suspension formulations intended to be administered using a syringe and needle, due to potential risk of needle clogging as a result of the formation of particle agglomerates. This may affect applicability of the formulation for administration by injection, as well as accurate dosing. Syringe glide force measurements of different injection systems were performed using a Texture Analyzer XT plus (Stable Micro Systems, Godalming, UK). Suspension formulations stored in vials were drawn into a 1 ml B. Braun Inject F single-use syringe (B. Braun AG, Melsungen, Germany) and a 27G Terumo Agani needle (Terumo, Tokyo, Japan) was then attached. For determination of glide forces needed for injection the plunger speed was set to obtain a volume flow of 0.1 ml/s. In order to investigate initial dispersion quality, injectability (27G needle) was tested manually. 27G needles have an approximate inner diameter of 210 μm.

### Results

Suspensions prepared utilizing an ultrasound bath or a high-shear homogenizer were generally found to be injectable when the spray dried and vacuum dried protein-containing particles prepared according to Example 1 were used to prepare the suspensions. In contrast, the use of protein-containing particles which did not undergo an additional vacuum drying step gave rise in some of the tested formulations to needle clogging as a consequence of an incomplete suspension.

For example, syringeability tests were conducted for suspension formulations 7a, 7b, 8a, 8b, (mAb:Suc 50:50; TSC=100 mg/ml) as described in Table 2, after storage over a 6-month period at 40° C. It was observed, that it was not possible to draw the suspensions 7a and 8a into a syringe using a 23G needle. No difficulties occurred when suspensions 7b and 8b containing mAb-particles were drawn into the syringe. Even after destroying the particle scaffold at a frequency of 30 Hz, the particles of suspension formulations 7a and 8a were observed to adhered to the vial wall and with poor dispersibility. As shown in Table 2, below, mostly air (as determined by removable volume) was drawn into the syringe:

TABLE 2

| | | | | | Residual | | |
|---|---|---|---|---|---|---|---|
| | | | | | water | | |
| | | | | | content, | | |
| For- | | | Sus- | Additional | suspended | | Removable |
| mulation, | | | pension | drying | particle | Storage | Volume |
| No. | Protein | Stabilizer | vehicle | step | [%] | conditions | [%] |
| 7a | mAb | sucrose | F4H5 | no | 4.2 | 6 | 4.70% |
| 7b | mAb | sucrose | F4H5 | yes | 0.1 | months | 92.00% |
| 8a | mAb | sucrose | F6H8 | no | 4.2 | at 40° C. | 4.30% |
| 8b | mAb | sucrose | F6H8 | yes | 0.1 | | 92.00% |

Thus, it appears that with the additional processing step comprising drying the protein particles (i.e. by vacuum-drying) may have an advantageous effect on the stability and applicability for use in injections.

### Lysozyme/Trehalose Suspensions

The injectability of suspension formulations comprising lysozyme-trehalose containing particles (lysozyme trehalose 70:30; TSC=100 mg/ml), formulations 1a, 1b, 1c, and 1d, as described in Table 1 stored over a period of 12 months at 40° C. was also tested according to the protocol described above. No significant changes in glide force, and no needle clogging was observed even after one year of storage at 40° C. for formulations in all the vehicles tested i.e. F4H5, F6H8, EO or MCT.

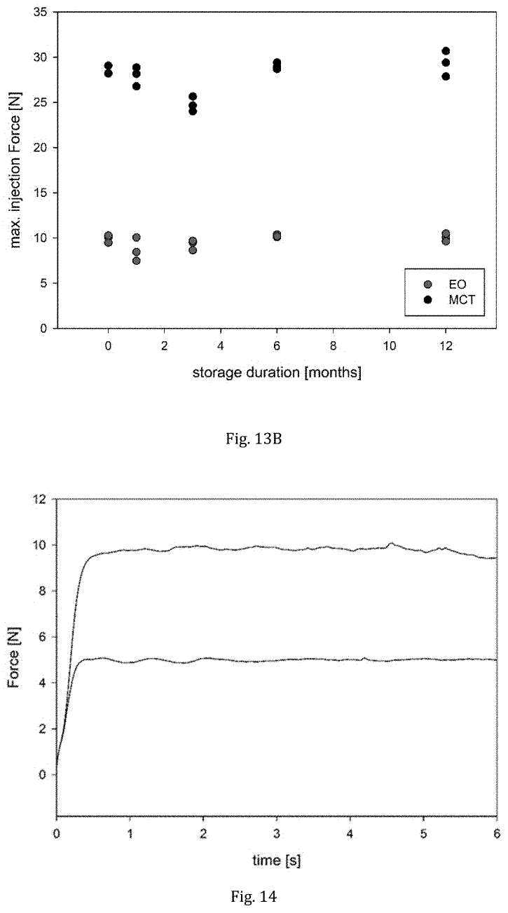

Similar results were obtained for lysozyme-trehalose suspension formulations with TSC=300 mg/ml and TSC=100 mg/ml (Lys:Tre 70:30; i.e. formulations 2, and 3 also containing polysorbate 20). FIG. 14 depicts the glide force profile of formulations 2 and 3 after 12 months of storage at 40° C.

Similar results were also obtained for the Lys:Tre 50:50 formulation in F6H8 (formulation 4, TSC=100 mg/mL).

Model mAB/Sucrose SUSPENSIONS

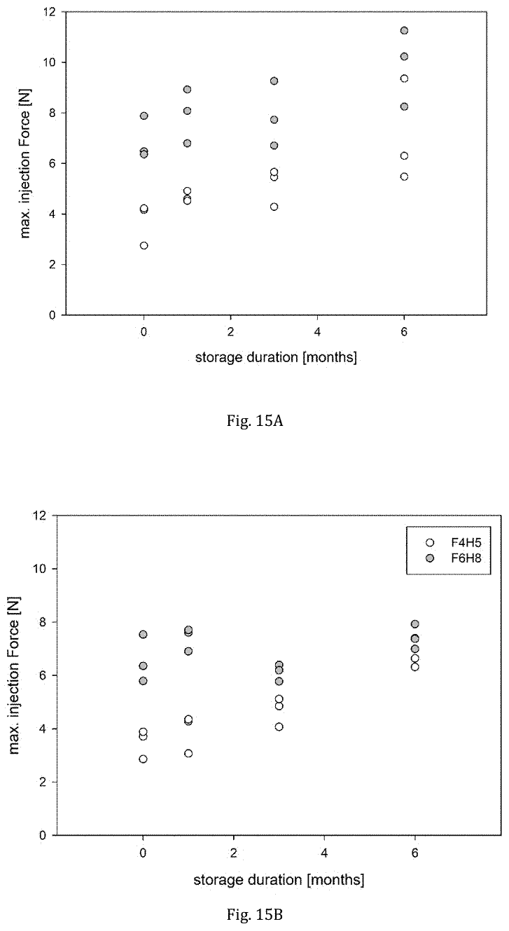

The injectability of suspension formulations with protein particles comprising model mAb-sucrose, 7a, 7b, (F4H5, mAb:Suc 50:50; TSC=100 mg/ml), and 8a, 8b (F6H8, mAb:Suc 50:50; TSC=100 mg/ml), as described in Table 1 stored over a period of 6 months at 40° C. was tested according to the protocol described above. FIGS. 15A and 15B depict maximum injection force required for injection of these formulations over a period of 6 months storage at 40° C. The formulations prepared with protein particles which were not subjected to the additional vacuum drying step, 7a, and 8a, i.e. the particles containing about 4.2 wt % of residual water content as depicted in FIG. 15A was observed to required increased application of force as storage at 40° C. progressed over the 6 month period. In comparison, the injectability results for the suspension formulations 7b and 8b prepared with protein particles which were vacuum dried after spray drying, and having a residual water content of about 0.1 wt % relative to weight of the particle appeared to remain consistent over the 6 month period (FIG. 15B).

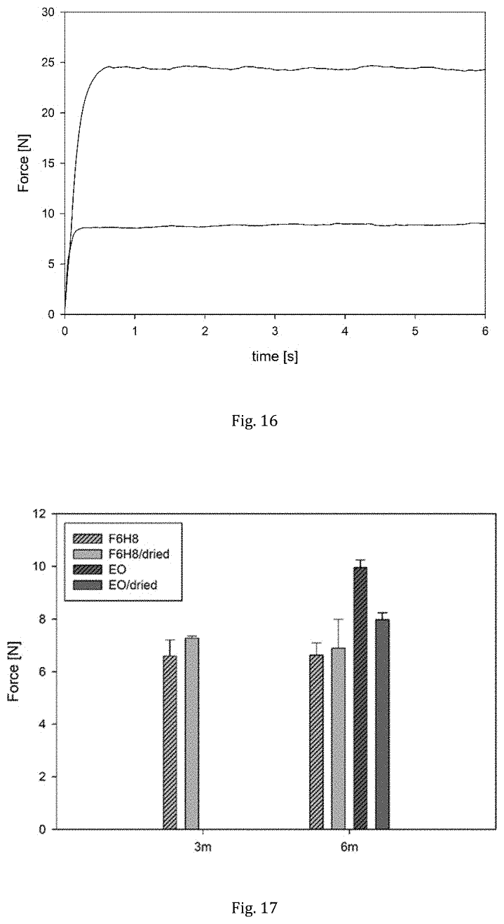

Similar results were obtained for mAb-sucrose suspension formulations prepared with the vehicles MCT (10b, mAb:suc 50:50, TSC=100 mg/mL) and EO (9b, mAb:suc 50:50, TSC=100 mg/mL). FIG. 16 depicts the glide force profile of formulations 9b and 10b after 6 months of storage at 40° C.

### Bevacizumab/Sucrose Suspensions

The injectability of suspension formulations with protein particles comprising bevacizumab-sucrose, 14a, 14b, (F6H8, beva:suc 50:50, TSC—100 mg/mL), and 15a, 15b (EO, beva:suc 50:50, TSC—100 mg/mL), as described in Table 1 which have been stored over a period of 6 months at 40° C. was tested according to the protocol described above. As shown in FIG. 17, bevacizumab-containing suspension formulations were found to be injectable, without any needle clogging or disturbances after 6 months of storage in both F6H8 and Ea

### Protein Activity Testing

ELISA was used to evaluate the activity of bevacizumab in formulation 16. Bevacizumab is involved in binding to VEGF which is associated with the inhibition of angiogenesis. The test is based on sandwich-type ELISA using a microtiter plate coated with recombinant human VEGF-A. Horseradish peroxidase (HRP)-conjugated anti-human IgG monoclonal antibody, which bind to the Fc region of antibodies, was employed to quantify the bound bevacizumab. ELISA-assays were performed using commercial kits from ImmunoGuide (Ankara, Turkey) according to the manufacturer's instructions utilizing aqueous commercial protein raw material and reconstituted protein suspension formulations.

The protein suspension formulations were prepared from spray-dried and subsequently vacuum-dried protein particles. The ELISA analyses showed no significant difference in the binding activity between the raw material and reconstituted protein suspension formulations. The process of protein particle preparation, subsequent drying as well as the subsequent preparation of the suspensions did not affect the activity of the anti-VEGF protein bevacizumab.

TABLE 3
Testing of bevacizumab suspension 16

| Temperature | Test | Method | Initial | 1 month | 3 months |
|---|---|---|---|---|---|
| 2-8° C. | Assay | UV- | 4.28 | 4.23 | 4.42 |
| | | Absorption | mg/ml | mg/ml | mg/ml |
| | Activity | ELISA | 91.5% | 99.3% | 94.7% |
| | Aggregation | SEC-MALS | 2.7% | 3.2% | 7.7% |
| 25° C./ | Assay | UV- | 4.28 | 4.20 | 4.33 |
| 65% RH | | Absorption | mg/ml | mg/ml | mg/ml |
| | Activity | ELISA | 91.5% | 92.1% | 85.3% |
| | Aggregation | SEC-MALS | 2.7% | 4.0% | 18.2% |

1. A method for the manufacture of a suspension formulation comprising a protein particle and a non-aqueous vehicle, the method comprising the steps of:
a) providing an aqueous solution comprising a protein and a stabilizing agent,
b) removing the water from said aqueous solution to obtain solid protein particles,
c) further drying the protein particles obtained in step b) to obtain protein particles comprising a residual water content of less than 0.5 wt %, based on the weight of the particle, and
d) suspending the protein particles of step c) in a non-aqueous vehicle comprising a semifluorinated alkane; and optionally;
e) homogenizing the suspension formulation, preferably by high-shear homogenization, milling, or ultrasonication;
wherein the protein particle comprises a protein and a stabilizing agent, and wherein the non-aqueous vehicle comprises a semifluorinated alkane.

2. The method according to claim 1, wherein the water in step b) is removed by spray-drying or lyophilizing the composition.

3. (canceled)

4. (canceled)

5. The method according to claim 1, wherein step c) drying is vacuum drying conducted at a temperature of between 15-40° C., at a pressure of between 0.01-100 mbar.

6. (canceled)

7. (canceled)

8. (canceled)

9. The method according to claim 1, wherein the relative weight ratio of the protein to the stabilizing agent in the protein particle is between 1:1 and 7:3.

10. The method according to claim 1, wherein the protein particles in step d) are resuspended in a semifluorinated alkane selected from F4H5 and F6H8.

11. The method according to claim 1, wherein the stabilizing agent is selected from saccharides, polyols, amino acids, amines, surfactants, antioxidants, polymers, salts or combinations thereof.

12. (canceled)

13. (canceled)

14. (canceled)

15. The method according to claim 1, wherein after step d) or optionally step e) at least 90% of the protein particles have a mean diameter of between 1 and 30 μm as determined by laser diffraction.

16. The method according to claim 1, wherein the protein concentration in the suspension formulation is between 2 and 350 mg/ml.

17. The method according to claim 1, wherein the total solid content of the suspension formulation is between 7 and 500 mg/ml.

18. (canceled)

19. The method according to claim 1, wherein the residual water content of the suspension formulation is less than 1.0 mg/ml based on the total volume of the formulation.

20. A suspension formulation comprising protein particles suspended in a non-aqueous vehicle, made according to the method of claim 1, wherein the protein particle comprises a protein and a stabilizing agent, and wherein the non-aqueous vehicle comprises a semifluorinated alkane.

21. A suspension formulation comprising twice dried protein particles comprising a protein and a stabilizing agent, suspended in a non-aqueous liquid vehicle comprising a semifluorinated alkane; wherein the residual water content of the protein particles is less than 0.5 wt %; and wherein at least 90% of the protein particles have a mean diameter of between 1 and 30 μm as determined by laser diffraction.

22. The suspension formulation according to claim 21, wherein the twice dried protein particle is spray-dried and vacuum dried or lyophilized and vacuum dried.

23. The suspension formulation according to claim 21, wherein the stabilizing agent is a saccharide, a polyol, a polysorbate or a combination thereof.

24. (canceled)

25. (canceled)

26. (canceled)

27. The suspension formulation according to claim 21, wherein the relative weight ratio of the protein to stabilizing agent in the protein particle is in the range of 1:1 to 7:3.

28. The suspension formulation according to claim 21, wherein the semifluorinated alkane is selected from F4H5 and F6H8.

29. The suspension formulation according to claim 21, wherein the protein concentration in the suspension formulations is between 2 and 350 mg/ml.

30. (canceled)

31. The suspension formulation according to claim 21, wherein the suspension formulation is free of a surfactant.

32. (canceled)

33. A kit comprising the suspension formulation of claim 20 and a container adapted for holding said formulation, and optionally a dispensing means.

34. An administration device comprising the suspension formulation of claim 20, wherein the administration device is adapted for topical administration, or administration of the suspension formulation by injection.

35. A kit comprising the suspension formulation of claim 21 and a container adapted for holding said formulation, and optionally a dispensing means.

36. An administration device comprising the suspension formulation of claim 21, wherein the administration device is adapted for topical administration, or administration of the suspension formulation by injection.
